(19) United States
(12) Patent Application Publication
(10) Pub. No.: US 2013/0137767 A1
(43) Pub. Date: May 30, 2013

(54) Methods and Compositions for Treating Arthritis with Docosahexaenoic Acid

(71) Applicants: Julie Nauroth, Odenton, MD (US); Melissa Olson, Pikesville, MD (US); Norman Salem, Rockville, MD (US); John Paul Zimmer, Boulder, CO (US)

(72) Inventors: Julie Nauroth, Odenton, MD (US); Melissa Olson, Pikesville, MD (US); Norman Salem, Rockville, MD (US); John Paul Zimmer, Boulder, CO (US)

(73) Assignee: DSM IP ASSETS B.V., TE Heerlen (NL)

(21) Appl. No.: 13/576,215

(22) PCT Filed: Feb. 2, 2011
(86) PCT No.: PCT/US11/23437

§ 371 Date: Feb. 8, 2013

(30) Foreign Application Priority Data
Feb. 2, 2010 (US) 61300823
Feb. 18, 2010 (US) 61305942

(51) Int. Cl. A61K 31/202; A61K 31/232

(52) U.S. Cl. CPC A61K 31/202; A61K 31/232
USPC 514/549; 514/560

(57) ABSTRACT

The present invention is directed to methods and compositions for treating arthritis in a subject, comprising administering about 430 mg to about 12.4 g of docosahexaenoic acid (DHA) per day in a dosage form to the subject in need thereof, wherein the dosage form is substantially free of eicosapentaenoic acid (EPA) and substantially free of non-alpha tocopherol.

### BACKGROUND OF THE INVENTION

1. Field of the Invention

The present invention is directed to methods and compositions for treating arthritis in a subject by administering docosahexaenoic acid (DHA) to the subject in need thereof, wherein the compositions are substantially free of eicosapentaenoic acid (EPA), and substantially free of non-alpha tocopherol.

2. Background

Polyunsaturated fatty acids (“PUFAs,” including long-chain PUFAs “LC-PUFAs”) have been reported to enhance cognitive function and to maintain cardiovascular health, among other benefits. In particular, omega-3 PUFAs have been reported to be important dietary components for preventing arteriosclerosis and coronary heart disease, for alleviating inflammatory conditions, and for retarding the growth of tumor cells, and omega-6 PUFAs are important as both structural lipids, and as precursors for, e.g., prostaglandins and leukotrienes.

Clinical trials have reported that fish oil supplementation benefits patients with rheumatoid arthritis. Two particular polyunsaturated fatty acids that have been reported to have a therapeutic efficacy of treating arthritis when used in combination are (all-Z)-5,8,11,14,17-eicosapentaenoic acid, hereinafter referred to as EPA, and (all-Z)-4,7,10,13,16,19-docosahexaenoic acid, hereinafter referred to as DHA. These LC-PUFAs are commonly found together in fatty fish, such as tuna, salmon, and mackerel.

### BRIEF SUMMARY OF THE INVENTION

Provided herein are methods for treating arthritis in a subject, comprising administering about 430 mg to about 12.4 g of docosahexaenoic acid (DHA) per day in a dosage form to the subject in need thereof wherein the dosage form is substantially free of eicosapentaenoic acid (EPA) and substantially free of non-alpha tocopherol.

In some embodiments, EPA is less than about 3% (w/w) of the total fatty acid content of the dosage form. In some embodiments, EPA is less than about 0.1% (w/w) of the total fatty acid content of the dosage form. In some embodiments, EPA is not detectable in the dosage form.

In some embodiments, the dosage form is substantially free of docosapentaenoic acid 22:5n-6 (DPAn6).

In some embodiments, the DHA is derived from an algal source. In some embodiments, the algal source is Crypthecodiunium cohnii, Thraustochytrium, or Schizochytrium sp.

In some embodiments, about 0.84 g to about 4 g of DHA is administered per day to the subject. In some embodiments, about 0.84 g to about 1.5 g of DHA is administered per day to the subject. In some embodiments, about 0.84 mg to about 1 g of DHA is administered per day to the subject.

In some embodiments, the DHA is administered once per day, twice per day, three times per day, four times per day, five times per day, six times per day, seven times per day, eight times per day, nine times per day, ten times per day, eleven times per day, or twelve times per day.

In some embodiments, the DHA is administered in a single dosage form. In some embodiments, the dosage form comprises about 0.4 g to about 1 g of DHA. In some embodiments, the dosage form has a total weight of about 0.2 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, about 1 g, or about 1.05 g.

In some embodiments, the DHA in the dosage form is about 30% to about 99.5% (w/w) of the total fatty acid content of the dosage form. In some embodiments, the DHA in the dosage form is about 35% to about 65% (w/w) of the total fatty acid content of the dosage form.

In some embodiments, the dosage form is characterized by one or more of the following amounts of fatty acids or esters thereof: (a) capric acid is about 1% or less (w/w); (b) lauric acid is about 1% or less (w/w); (c) myristic acid is about 1% (w/w) or less; (d) palmitic acid is about 1% or less (w/w); (e) palmitoleic acid is about 1% (w/w) or less; (f) stearic acid is about 1% (w/w) or less; (g) oleic acid is about 1% (w/w) or less; (h) linoleic acid is about 1% (w/w) or less; (i) α-linolenic acid is about 1% (w/w) or less; (j) docosapentaenoic acid 22:5n-3 (DPAn3) is about 1% (w/w) or less; (k) docosapentaenoic acid 22:5n-6 (DPAn6) is about 1% or less (w/w); and (l) 4,7,10,13,16,19,22,25 octacosaoctaenoic acid (C28:8) is about 1% (w/w) or less of the total fatty acid content of the dosage form.

In some embodiments, the DHA in the dosage form comprises about 40% to about 50% (w/w) of the total weight of the dosage form. In some embodiments, the DHA in the dosage form comprises about 40% to about 45% (w/w) of the total fatty acid content of the dosage form.

In some embodiments, the dosage form is characterized by one or more of the following amounts of fatty acid or esters thereof: (a) capric acid (C10:0) is about 2% or less; (b) lauric acid (C12:0) is about 6% or less; (c) myristic acid (C14:0) is about 20% or less; (d) palmitic acid (C16:0) is about 20% or less; (e) palmitoleic acid (C16:1n-7) is about 3% or less; (f) stearic acid (C18:0) is about 2% or less; (g) oleic acid (C18:1n-9) is about 40% or less; (h) linoleic acid (C18:2) is about 5% or less; (i) arachidonic acid (C20:4) is about 0.1% or less; (j) eicospentaenoic acid (C20:5) is about 3% or less; (k) decosapentaenoic acid (22:5n-6) is about 0.1% or less; (l) nervonic acid (C24:1) is about 2% or less; and (m) others is about 3% or less of the total fatty acid content of the dosage form.

In some embodiments, the DHA in the dosage form comprises about 35% to about 45% (w/w) of the total weight of the dosage form. In some embodiments, the DHA in the dosage form comprises about 35% to about 45% (w/w) of the total fatty acid content of the dosage form.

In some embodiments, the dosage form is characterized by one or more of the following amounts of fatty acid or esters thereof: (a) capric acid (C10:0) is about 0.01% or less; (b) lauric acid (C12:0) is about 0.5% or less; (c) myristic acid (C14:0) is about 12% or less; (d) palmitic acid (C16:0) is about 28% or less; (e) palmitoleic acid (C16:1n-7) is about 0.5% or less; (f) stearic acid (C18:0) is about 2% or less; (g) oleic acid (C18:1n-9) is about 8% or less; (h) linoleic acid (C18:2) is about 2% or less; (i) arachidonic acid (C20:4) is about 2% or less; (j) eicospentaenoic acid (C20:5) is about 3% or less; (k) decosapentaenoic acid (22:5n-6) is about 18% or less; (l) nervonic acid (C24:1) is about 0.01% or less; and (m) others is about 10% or less; of the total fatty acid content of the dosage form.

In some embodiments, the DHA in the dosage form comprises about 55% to about 57% (w/w) of the total weight of the dosage form. In some embodiments, the DHA in the dosage form comprises about 55% to about 67% (w/w) of the total weight of the dosage form. In some embodiments, the DHA in the dosage form comprises about 55% to about 67% (w/w) of the total fatty acid content of the dosage form.

In some embodiments, the dosage form is characterized by one or more of the following amounts of fatty acid or esters thereof: (a) capric acid (C10:0) is about 2% or less; (b) lauric acid (C12:0) is about 6% or less; (c) myristic acid (C14:0) is about 20% or less; (d) palmitic acid (C16:0) is about 15% or less; (e) palmitoleic acid (C16: 1n-7) is about 5% or less; (f) stearic acid (C18:0) is about 2% or less; (g) oleic acid (C18:1n-9) is about 20% or less; (h) linoleic acid (C18:2) is about 2% or less; (i) arachidonic acid (C20:4) is about 0.1% or less; (j) eicospentaenoic acid (C20:5) is about 0.1% or less; (k) decosapentaenoic acid (22:5n-6) is about 0.1% or less; and (l) others is about 3% or less; of the total fatty acid content of the dosage form.

In some embodiments, the DHA comprises about 67% to about 72% (w/w) of the total fatty acid content of the dosage form.

In some embodiments, the DHA in the dosage form comprises about 85% to about 96% (w/w) of the total weight of the dosage form. In some embodiments, the DHA in the dosage form comprises about 85% to about 99.5% (w/w) of the total fatty acid content of the dosage form.

In some embodiments, the dosage form is characterized by one or more of the following amounts of fatty acid or esters thereof: (a) capric acid (C10:0) is about 0.1% or less; (b) lauric acid (C12:0) is about 0.1% or less; (c) myristic acid (C14:0) is about 0.1% or less; (d) palmitic acid (C16:0) is about 0.5% or less; (e) palmitoleic acid (C16:1n-7) is about 0.5% or less; (f) stearic acid (C18:0) is about 0.5% or less; (g) oleic acid (C18:1n-9) is about 4% or less; (h) linoleic acid (C18:2) is about 0.1% or less; (i) arachidonic acid (C20:4) is about 0.1% or less; (j) eicospentaenoic acid (C20:5) is about 0.1% or less; (k) decosapentaenoic acid (22:5n-6) is about 3% or less; and (l) others is about 1% or less of the total fatty acid content of the dosage form.

In some embodiments, the DHA is in the form of an ester. In some embodiments, the DHA is in the form of a triglyceride. In some embodiments, the ester is an alkyl ester. In some embodiments, the alkyl ester is a methyl ester, ethyl ester, propyl ester, or combinations thereof. In some embodiments, the dosage form, particularly at least one unit dose, and more particularly a gelatin capsule, comprises about 430 mg to about 480 mg of DHA ethyl ester. In some embodiments, the dosage form, particularly at least one unit dose, and more particularly a gelatin capsule, comprises about 860 mg to about 950 mg of DHA ethyl ester. In some embodiments, the dosage form, particularly at least one unit dose, and more particularly a gelatin capsule, comprises about 870 mg to about 930 mg of DHA ethyl ester.

In some embodiments, the dosage form is administered once per day. In some embodiments, the dosage form is administered once per day for the remainder of the subject's lifetime. In some embodiments, the dosage form is administered once per day for about 1 year to about 10 years. In some embodiments, the dosage form is administered once per day for about 1 month to about 12 months. In some embodiments, the dosage form is administered once per day for at least 6 months.

In some embodiments, the dosage form is an oral dosage form. In some embodiments, the oral dosage form is a gelatin capsule or a caplet. In some embodiments, the oral dosage form is a gelatin capsule. In some embodiments, the gelatin capsule is a soft gelatin capsule. In some embodiments, the gelatin capsule comprises a plasticizer, gelatin, and water. In some embodiments, the plasticizer is glycerol or glycerin.

In some embodiments, the arthritis is selected from the group consisting of osteoarthritis, gouty arthritis, ankylosing spondylitis, psoriatic arthritis, reactive arthritis, rheumatoid arthritis, juvenile onset rheumatoid arthritis, infectious arthritis, inflammatory arthritis, septic arthritis, degenerative arthritis, arthritis mutilans, and lyme arthritis

In some embodiments, the dosage form is administered in combination with an agent selected from the group consisting of nonsteroidal anti-inflammatory drugs (NSAIDs), cox-2 inhibitors, steroids, narcotic pain relievers, disease-modifying antirheumatic drugs (DMARDs), TNF blockers, biological response modifiers, chemotherapy medications, and gout medicines.

The present invention is also directed to an oral dosage form consisting essentially of about 430 mg to about 480 mg of docosahexaenoic acid (DHA), wherein the dosage form is substantially free of eicosapentaenoic acid (EPA) and substantially free of non-alpha tocopherol.

The present invention is also directed to an oral dosage form consisting essentially of about 860 mg to about 950 mg of docosahexaenoic acid (DHA), wherein the dosage form is substantially free of eicosapentaenoic acid (EPA) and substantially free of non-alpha tocopherol.

In some embodiments, the dosage form is a gelatin capsule. In some embodiments, the gelatin capsule is a soft gelatin capsule. In some embodiments, the gelatin capsule comprises a plasticizer, gelatin, and water. In some embodiments, the plasticizer is glycerol or glycerin. In some embodiments, the dosage form comprises about 860 mg to about 950 mg DHA. In some embodiments, the dosage form comprises about 870 mg to about 930 mg DHA. In some embodiments, the DHA is derived from an algal source.

The present invention is also directed to a kit comprising a dosage form and a label containing instructions to administer the dosage form once per day, twice per day, three times per day, four times per day, five times per day, six times per day, seven times per day, eight times per day, nine times per day, ten times per day, eleven times per day, or twelve times per day, to a subject in need thereof.

### BRIEF DESCRIPTION OF THE FIGURES

FIG. 1 shows the arthritis severity scores for days 18-45 following the specified fatty acid ethyl ester treatment at 2500 mg/kg (A) and 5000 mg/kg (B) in male DBA/1OaHsd mice having collagen-induced arthritis, a chronic mouse model of inflammation.

FIG. 2 shows the arthritis severity score, presented as area under the curve (AUC), for days 18-45 following the specified fatty acid ethyl ester treatment at 2500 mg/kg and 5000 mg/kg in male DBA/1OaHsd mice having collagen-induced arthritis.

FIG. 3 shows the arthritis incidence for days 18-45 following the specified fatty acid ethyl ester treatment at 2500 mg/kg (A) and 5000 mg/kg (B) in male DBA/1OaHsd mice having collagen-induced arthritis.

FIG. 4 shows the paw, knee and joint histopathology parameters following the specified fatty acid ethyl ester treatment at 2500 mg/kg and 5000 mg/kg in male DBA/1OaHsd mice having collagen-induced arthritis.

FIG. 5 shows serum phospholipid fatty acid levels (DHA, DPAn6, EPA and ARA) following the specified fatty acid ethyl ester treatment at 2500 mg/kg and 5000 mg/kg in male DBA/1OaHsd mice having collagen-induced arthritis.

FIG. 6 shows anti-collagen antibody production levels at day 21 (A) and day 45 (B) following the specified fatty acid ethyl ester treatment at 2500 mg/kg in male DBA/1OaHsd mice having collagen-induced arthritis.

FIG. 7 shows arthritis severity scores following triglyceride (A) or ethyl ester (B) treatment with the specified fatty acid in male DBA/1OaHsd mice having collagen-induced arthritis.

FIG. 8 shows the arthritis severity score, presented as area under the curve (AUC), for days 18-45 following (A) triglyceride or (B) ethyl ester treatment with the specified fatty acid in male DBA/1OaHsd mice having collagen-induced arthritis.

FIG. 9 shows the arthritis incidence for days 18-45 following the specified fatty acid triglyceride (A) or ethyl ester (B) treatment in male DBA/1OaHsd mice having collagen-induced arthritis.

FIG. 10 shows the paw, knee and joint histopathology parameters following triglyceride and ethyl ester treatment with the specified fatty acid in male DBA/1OaHsd mice having collagen-induced arthritis.

FIG. 11 shows serum phospholipid fatty acid levels (DHA, EPA, DPAn6 and ARA) following triglyceride (column 1) or ethyl ester (column 2) treatment in male DBA/1OaHsd mice having collagen-induced arthritis.

FIG. 12 shows anti-collagen antibody production levels at day 21 (A) and day 45 (B) following triglyceride and ethyl ester treatment in male DBA/1OaHsd mice having collagen-induced arthritis.

### DETAILED DESCRIPTION OF THE INVENTION

For the descriptions herein and the appended claims, the singular forms “a” “an” and “the” include plural referents unless the context clearly indicates otherwise. Thus, for example, reference to “a compound” refers to more than one compound.

Also the use of “or” means “and/or” unless stated otherwise. Similarly, “comprise,” “comprises,” “comprising,” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

In reference to the present disclosure, the technical and scientific terms used in the descriptions herein will have the meanings commonly understood by one of ordinary skill in the art, unless specifically defined otherwise.

The present invention is directed to methods for treating arthritis in a subject, comprising administering about 430 mg to about 12.4 g of docosahexaenoic acid per day in a dosage form to the subject in need thereof, wherein the dosage form is substantially free of eicosapentaenoic acid and substantially free of non-alpha tocopherol.

“DHA” refers to docosahexaenoic acid, also known by its chemical name (all-Z)-4,7,10,13,16,19-docosahexaenoic acid, as well as any salts or derivatives thereof. Thus, the term “DHA” encompasses the free acid DHA as well as DHA phospholipids, esters, monoglycerides, diglycerides, and triglycerides containing DHA. DHA is an ω-3 polyunsaturated fatty acid.

The DHA can be in a mono, di, or triglyceride form. For example, one, two or three DHA molecules can be in the mono, di or triglyceride molecule.

In some embodiments, the DHA of the present invention is an ester. The term “ester” herein refers to the replacement of the hydrogen in the carboxylic acid group of the DHA molecule with another substituent. Typical esters are known to those in the art. Examples of the most common esters include methyl, ethyl, propyl, butyl, pentyl, t-butyl, benzyl, nitrobenzyl, methoxybenzyl, benzhydryl, and trichloroethyl. In some embodiments, the ester is a carboxylic acid protective ester group, esters with aralkyl (e.g., benzyl, phenethyl), esters with lower alkenyl (e.g., allyl, 2-butenyl), esters with lower-alkoxy-lower-alkyl (e.g., methoxymethyl, 2-methoxyethyl, 2-ethoxyethyl), esters with lower-alkanoyloxy-lower-alkyl (e.g., acetoxymethyl, pivaloyloxymethyl, 1-pivaloyloxyethyl), esters with lower-alkoxycarbonyl-lower-alkyl (e.g., methoxycarbonylmethyl, isopropoxycarbonylmethyl), esters with carboxy-lower alkyl (e.g., carboxymethyl), esters with lower-alkoxycarbonyloxy-lower-alkyl (e.g., 1-ethoxycarbonyloxy)ethyl, 1-(cyclohexyloxycarbonyloxy)ethyl), esters with carbamoyloxy-lower alkyl (e.g., carbamoyloxymethyl), and the like. In some embodiments, the added substituent is a linear or cyclic hydrocarbon group, e.g., a C₁-C₆ alkyl, C₁-C₆ cycloalkyl, C₁-C₆ alkenyl, or C₁-C₆ aryl ester.

In some embodiments, the ester is an alkyl ester, e.g., a methyl ester, ethyl ester or propyl ester. More particularly, the ester is an ethyl ester. The ester substituent can be added to the DHA free acid molecule when the DHA is in a purified or semi-purified state. Alternatively, the DHA ester is formed upon conversion of a triglyceride to a ester. One of skill in the art can appreciate that some non-esterified DHA molecules can be present in the present invention, e.g., DHA molecules that have not been esterified, or DHA linkages that have been cleaved, e.g., hydrolyzed. In some embodiments, the non-esterified DHA molecules constitute less than 3% (mol/mol), about 2% to about 0.01% (mol/mol), about 1% to about 0.05% (mol/mol), or about 5% to about 0.1% (mol/mol) of the total DHA molecules. Alternatively, in some embodiments, the DHA of the present invention can be in a free acid form and/or in a salt form.

As used herein, the terms “substantially free of EPA” and “substantially free of DPAn6” refer to a dosage form that does not contain an amount of EPA or DPAn6 sufficient to inhibit the inflammation-reducing action of DHA. The term “substantially free” of a fatty acid may also refer to a level below which biological effects following administration of a composition cannot be attributed to the presence of the fatty acid. As used herein, “or less” or “less than about” refers to percentages that include 0%, or amounts not detectable by current means. As used herein, “max” refers to percentages that include 0%, or amounts not detectable by current means.

In some embodiments, the dosage form is substantially free of eicosapentaenoic acid (EPA). EPA refers to eicosapentaenoic acid, known by its chemical name (all-Z)-5,8,11,14,17-eicosapentaenoic acid, as well as any salts or derivatives thereof. Thus, the term “EPA” encompasses the free acid EPA as well as EPA alkyl esters and triglycerides containing EPA. EPA is an ω-3 polyunsaturated fatty acid. As used herein, the term “substantially free of EPA” may refer to a dosage form in which EPA is less than about 3% (w/w) of the total fatty acid content of the dosage form. In some embodiments, the dosage form comprises, less than about 2% EPA (w/w) of the total fatty acid content of the dosage form, less than about 1% EPA (w/w) of the total fatty acid content of the dosage form, less than about 0.5% EPA (w/w) of the total fatty acid content of the dosage form, less than about 0.2% EPA (w/w) of the total fatty acid content of the dosage form, or less than about 0.01% EPA (w/w) of the total fatty acid content of the dosage form. In some embodiments, the dosage form has no detectable amount of EPA using techniques known in the art. In some embodiments, the DHA composition has no EPA.

In some embodiments of the method, the composition used has a level of DHA that is at least 40 wt % of total wt of fatty acid content. In some embodiments, the weight % of the DHA in the composition of DHA is at least 50 wt % of total wt of fatty acid content, at least 60 wt % of total wt of fatty acid content; at least 70 wt % of total wt of fatty acid content, at least 80 wt % of total wt of fatty acid content; at least 85 wt % of total wt of fatty acid content; at least 90 wt % of total wt of fatty acid content; at least 95 wt % of total wt of fatty acid content; at least 96 wt % of total wt of fatty acid content; at least 97 wt % of total wt of fatty avid content; at least 98 wt % of total wt of fatty acid content; or at least 99 wt % of total wt of fatty acid content.

In some embodiments, DHA can also be substantially free of docosapentaenoic acid 22:5n-6 (DPAn6). In some embodiments, the term “substantially free of DPAn6” refers to a dosage form comprising less than about 2% docosapentaenoic acid 22:5n-6 (DPAn6) (w/w) of the total fatty acid content of the dosage form. In some embodiments, the dosage form comprises less than about 1% DPAn6 (w/w) of the total fatty acid content of the dosage form. In some embodiments, the dosage form comprises less than about 0.5% (w/w) of the total fatty acid content of the dosage form. In some embodiments, the dosage form does not comprise any detectable amount of DPAn6.

In some embodiments, the composition of DHA may include an additional lipid. As used herein, the term “lipid” includes phospholipids (PL); free fatty acids; esters of fatty acids; triacylglycerols (TAG); diacylglycerides; monoacylglycerides; phosphatides; waxes (esters of alcohols and fatty acids); sterols and sterol esters; croetnoids; xanthophylls (e.g., oxycarotenoids); hydrocarbons; and other lipids known to one of ordinary skill in the art. The lipid can be chosen to have minimal adverse health effects or minimally affect the effectiveness of DHA when administered in combination with DHA.

In some embodiments, the composition of DHA may include an additional unsaturated lipid. In some embodiments, the unsaturated lipid is a polyunsaturated lipid, such as an omega-3 fatty acid or omega-6 fatty acid. An exemplary omega-6 fatty acid that may be used in the composition is docosapentaenoic acid (DPA), including DPA (n-6) or DPA (n-3).

The dosage form can also be substantially free of arachidonic acid (ARA). ARA refers to the compound (all-Z)-5,8,11,14-eicosatetraenoic acid (also referred to as (5Z,8Z,11Z,14Z)-icosa-5,8,11,14-tetraenoic acid), as well as any salts or derivatives thereof. Thus, the term “ARA” encompasses the free acid ARA as well as ARA alkyl esters and triglycerides containing ARA. ARA is an 0-6 polyunsaturated fatty acid. In some embodiments, the ARA is less than about 3% (w/w) of the total fatty acid content of the dosage form. In some embodiments, ARA comprises less than about 2% of the total fatty acid content of the dosage form, less than about 1% of the total fatty acid content of the dosage form, less than about 0.5% of the total fatty acid content of the dosage form, less than about 0.2% of the total fatty acid content of the dosage form, or less than about 0.01% of the total fatty acid content of the dosage form. In some embodiments, the dosage form has no detectable amount of ARA.

As used herein, the terms “substantially free of a flavonoid” and “substantially free of non-alpha tocopherol” mean that the dosage form comprises an amount of a flavonoid or non-alpha tocopherol that is insufficient for the flavonoid or non-alpha tocopherol to be considered an active agent. In some embodiments, a flavonoid may be present in the dosage form in an amount less than about 10 mg, less than about 5 mg, or less than about 1 mg. In some embodiments, the dosage form has no detectable amount of a flavonoid. In some embodiments, non-alpha tocopherol may be present in the dosage form in an amount less than about 50 mg, less than about 40 mg, less than about 30 mg, less than about 20 mg, less than about 10 mg, less than about 5 mg, or less than about 1 mg. In some embodiments, the dosage form has no detectable amount of non-alpha tocopherol.

In some embodiments, the dosage form is substantially free of a flavonoid. By a “flavonoid” is meant any of a class of polyphenolic molecules (including hesperetin and derivatives thereof) based on a flavan nucleus, comprising 15 carbon atoms, arranged in three rings as C₆-C₃-C₆. Flavonoids are generally classified into subclasses by the state of oxidation and the substitution pattern at the C₂-C₃ unit. As used herein, the term “flavonoid” encompasses, but are not limited to, flavanones, flavonols, flavones, anthocyanidins, chalcones, dihydrochalcones, aurones, flavanols, dihydroflavanols, proanthocyanidins (flavan-3,4-diols), isoflavones and neoflavones.

As used herein, the term “flavonoids” encompasses, but is not limited to: chrysin (5,7-dihydroxy-2-phenyl-4H-1-benzopyran-4-one; 5,7-dihydroxyflavone, chrysidenon); daidzein (7-hydroxy-3-(4-hydroxyphenyl)4H-1-benzopyran-4-one; 4′,7-dihydroxyisoflavone); diosmin(7-[[6-O-6-deoxy-α-L-mannopyranosyl)-β-D-glucopyranosyl]oxy]-5-hydroxy-2-(3-hydroxy-4-methoxyphenyl)4H-1-benzopyran-4-one); 3′,5,7-trihydroxy-4′-methoxyflavone-7-rutinoside; 5-hydroxy-2-(3-hydroxy-4-methoxyphenyl)-7-(O⁶-α-L-rhamnopyranosyl-β-D-glucopyranosyloxy)chromen-4-one; 5-hydroxy-2-(3-hydroxy-4-methoxyphenyl)-7-β-rutinosyloxy-4H-chromen-4-one; diosmetin; 7-β-rutinoside; barosmin; buchu resin; Daflon; Diosmil; Diovenor; Flebopex; Flebosmil; Flebosten; Flebotropin; Hemerven; Insuven; Tovene; Varinon; Ven-Detrex; Venex; Veno-V; Venosmine; hesperetin ((S)-2,3-dihydro-5,7-dihydroxy-2-(3-hydroxy-4-methoxyphenyl)4H-1-benzopyran-4-one); 3′,5,7-trihydroxy-4′-methoxyflavanone; cyanidenon 4′-methyl ether 1626; hesperidin ((S)-7-[[6-O-(6-deoxy-α-L-mannopyranosyl)-β-D-glucopyranosyl]oxy]-2,3-dihydro-5-hydroxy-2-(3-hydroxy-4-methoxyphenyl)4H-1-benzopyran-4-one); hesperetin (7-rhamnoglucoside); cirantin; hesperetin-7-rutinoside; luteolin (2-(3,4-dihydroxyphenyl)-5,7-dihydroxy-4H-1-benzopyran-4-one); 3′,4′,5,7-tetrahydroxyflavone; digitoflavone; cyanidenon 1470; quercetin (2-(3,4-dihydroxyphenyl)-3,5,7-trihydroxy-4H-1-benzopyran-4-one); 3,3′,4′,5,7-pentahydroxyflavone; memtin; sophoretin; cyanidenolon 1522; rutin (3-[[6-O-(6-deoxy-α-L-mannopyranosyl)-β-D-glucopyranosyl]oxy]-2-(3,4-dihydroxyphenyl)-5,7-dihydroxy-4H-1-benzopyran-4-one); rutoside; quercetin-3-rutinoside; 3,3′,4′,5,7-pentahydroxyflavone-3-rutinoside; melin; phytomelin; eldrin; ilixathin; sophorin; globularicitrin; paliuroside; osyritrin; osyritin; myrticolorin; violaquercitrin; Birutan; Rutabion; Rutozyd; Tanrutin; biochanin or biochanin A (5,7-dihydroxy-4′-methoxyiso-flavone); and olmelin.

In some embodiments, the dosage form is substantially free of non-alpha tocopherol. By “tocopherol” is meant any of a family of molecules which are characterized by a 6-chromanol ring structure and a side chain at the 2 position. A “non-alpha-tocopherol enriched tocopherol composition,” as used herein refers to the non-alpha-tocopherol, such as for example, gamma-, beta- or delta-tocopherol as being enriched with respect to total tocopherols in the composition. Tocopherols possess a 4′,8′,12′-trimethyltridecyl phytol side chain. As used herein, the term “tocopherol” encompasses, but is not limited to: alpha-tocopherol, beta-tocopherol, gamma-tocopherol, delta-tocopherol, epsilon-tocopherol, [R-(E,E)]-3,4-dihydro-2,5,8-trimethyl-2-(4,8,12-trimethyl-3,7,11-tridecatrienyl)-2H-1-benzopyran-6-ol; 2,5,8-trimethyl-2-(4,8,12-trimethyltrideca-3-,7,11-trienyl)chroman-6-ol; 5-methyltocol; zeta₁-tocopherol, 3,4-dihydro-2,5,7,8-tetramethyl-2-(4,8,12-trimethyl-3,7,11-tridecatrienyl-)-2H-1-benzopyran-6-ol; 2,5,7,8-tetramethyl-2-(4,8,12-trimethyl-3,7,11-tri-decatrienyl)-6-chromanol; 5,7,8-trimethyltocotrien-3′,7′,11′-ol; zeta₂-tocopherol, 3,4-dihydro-2,5,7-trimethyl-2-(4,8,12-trimethyltri-decyl)-2H-1-benzopyran-6-ol; 2,5,7-trimethyl-2-(4,8,12-trimethyltridecyl-6-chromanol; 5,7-dimethyltocol; and eta-tocopherol, 3,4-dihydro-2,7-dimethyl-1-2-(4,8,12-trimethyltridecyl)-2H-1-benzopyran-6-ol; 2,7-dimethyl-2-(4,8,12-trimethyltridecyl)-6-chromanol; 7-methyltocol. Other tocopherols include xi₁-, xi₂-, and sigma-tocopherols.

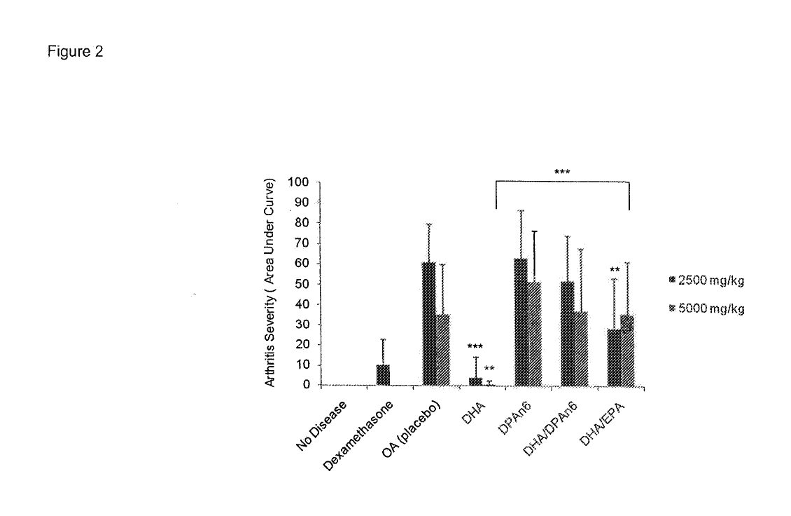

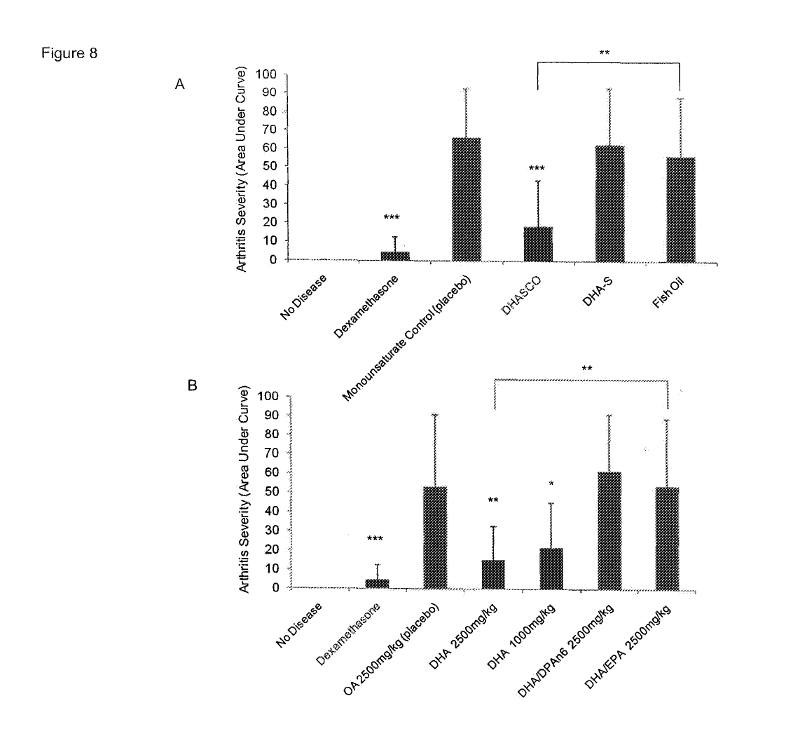

The presence of EPA was found to inhibit DHA-mediated reduction of joint inflammation in an animal model. It has been determined that DHA, substantially free of EPA or docosapentaenoic acid 22:5n-6 (DPAn6), causes a significant decrease in the arthritic score of mice with collagen induced arthritis. However, when a similar amount of DHA is used in combination with either DPAn6 or EPA, the arthritic score of mice with collagen-induced arthritis does not decrease, thus suggesting that DHA, free of EPA or DPAn6, is a superior treatment for arthritis. See FIG. 2 (DHA administered at 2500 mg/kg and 5000 mg/kg; DHA/DPAn6 administered at 1785 mg/kg DHA and 714 mg/kg DPAn6 (total 2500 mg/kg) and 3,571 mg/kg DHA and 1,428 mg/kg DPAn6 (total 5000 mg/kg); and DHA/EPA administered at 1000 mg/kg DHA and 1500 mg/kg EPA (total 2500 mg/kg) and 2000 mg/kg DHA and 3000 mg/kg EPA (total 5000 mg/kg)) and FIG. 8 (DHA administered at 1000 mg/kg and 2500 mg/kg; DHA/DPAn6 administered at 1785 mg/kg DHA and 714 mg/kg DPAn6; and DHA/EPA administered at 1000 mg/kg DHA and 1500 mg/kg EPA).

The term DPAn6 refers to docosapentaenoic acid, an omega 6 fatty acid, known by its chemical name (all-Z)-4,7,10,13,16-docosapentaenoic acid, as well as any salts or esters thereof. Thus, the term DPAn6 encompasses the free acid DPAn6, as well as DPAn6 alkyl esters and triglycerides containing DPAn6. DPAn6 can be removed during purification of DHA, or alternatively, the DHA can be obtained from an organism that does not produce DPAn6, or produces very little DPAn6. In some embodiments, the dosage form comprises less than about 2% DPAn6 or less than about 1% DPAn6 (w/w) of the total fatty acid content of the dosage form. In some embodiments, DPAn6 is not detectable in the dosage form using techniques currently known in the art.

The DHA of the present invention can be derived from various sources, e.g., from oleaginous microorganisms. As used herein, “oleaginous microorganisms” are defined as microorganisms capable of accumulating greater than 20% of the dry weight of their cells in the form of lipids. In some embodiments, the DHA is derived from a phototrophic or heterotrophic single cell organism or multicellular organism, e.g., an algae. In some embodiments, the DHA can be derived from a diatom, e.g., a marine dinoflagellates (algae), such as Crypthecodinium sp., Thraustochytrium sp., Schizochytrium sp., or combinations thereof.

The source of the DHA can include a microbial source, including the microbial groups Stramenopiles, Thraustochytrids, and Labrinthulids. Stramenopiles includes microalgae and algae-like microorganisms, including the following groups of microorganisms: Hamatores, Proteromonads, Opalines, Develpayella, Diplophrys, Labrinthulids, Thraustochytrids, Biosecids, Oomycetes, Hypochytridiomycetes, Commation, Reticulosphaera, Pelagomonas, Pelagococcus, Ollicola, Aureococcus, Parmales, Diatoms, Xanthophytes, Phaeophytes (brown algae), Eustigmatophytes, Raphidophytes, Synurids, Axodines (including Rhizochromulinaales, Pedinellales, Dictyochales), Chrysomeridales, Sarcinochrysidales, Hydrurales, Hibberdiales, and Chromulinales. The Thraustochytrids include the genera Schizochytrium (species include aggregatum, limnaceum, mangrovei, minutum, octosporum), Thraustochytrium (species include arudimentale, aureum, benthicola, globosum, kinnei, motivum, multirudimentale, pachydermum, proliferum, roseum, striatum), Ulkenia (species include amoeboidea, kerguelensis, minuta, profunda, radiate, sailens, sarkariana, schizochytrops, visurgensis, yorkensis), Aplanochytrium (species include haliotidis, kerguelensis, profunda, stocchinoi), Japonochytrium (species include marinum), Althornia (species include crouchii), and Elina (species include marisalba, sinorifica). The Labrinthulids include the genera Labyrinthula (species include algeriensis, coenocystis, chattonii, macrocystis, macrocystis atlantica, macrocystis macrocystis, marina, minuta, roscoffensis, valkanovii, vitellina, vitellina pacifica, vitellina vitellina, zopfi), Labyrinthomyxa (species include marina), Labyrinthuloides (species include haliotidis, yorkensis), Diplophrys (species include archeri), Pyrrhosorus* (species include marinus), Sorodiplophrys* (species include stercorea), and Chlamydomyxa* (species include labyrinthuloides, montana) (*=there is no current general consensus on the exact taxonomic placement of these genera).

In some embodiments, the algal source is, e.g., Crypthecodinium cohnii. Samples of C. cohnii, have been deposited with the American Type Culture Collection at Rockville, Md., and assigned accession nos. 40750, 30021, 30334-30348, 30541-30543, 30555-30557, 30571, 30572, 30772-30775, 30812, 40750, 50050-50060, and 50297-50300.

As used herein, the term microorganism, or any specific type of organism, includes wild strains, mutants or recombinant types. Organisms which can produce an enhanced level of oil containing DHA are considered to be within the scope of this invention. Also included are microorganisms designed to efficiently use more cost-effective substrates while producing the same amount of DHA as the comparable wild-type strains. Cultivation of dinoflagellates such as C. cohnii has been described previously. See, U.S. Pat. No. 5,492,938 and Henderson et al., Phytochemistry 27:1679-1683 (1988). Organisms useful in the production of DHA can also include any manner of transgenic or other genetically modified organisms, e.g., plants, grown either in culture fermentation or in crop plants, e.g., cereals such as maize, barley, wheat, rice, sorghum, pearl millet, corn, rye and oats; or beans, soybeans, peppers, lettuce, peas, Brassica species (e.g., cabbage, broccoli, cauliflower, brussel sprouts, rapeseed, and radish), carrot, beets, eggplant, spinach, cucumber, squash, melons, cantaloupe, sunflowers, safflower, canola, flax, peanut, mustard, rapeseed, chickpea, lentil, white clover, olive, palm, borage, evening primrose, linseed, and tobacco.

Another source of oils containing DHA suitable for the compositions and methods of the present invention includes an animal source. Examples of animal sources include aquatic animals (e.g., fish, marine mammals, and crustaceans such as krill and other euphausids) and animal tissues (e.g., brain, liver, eyes, etc.) and animal products such as eggs or milk. Thus, in some embodiments, the method of the present invention comprises administering daily to the subject a dosage form comprising docosahexaenoic acid (DHA) substantially free of eicosapentaenoic acid (EPA), wherein the DHA is derived from a non-algal source, e.g., fish.

DHA can be purified to various levels. DHA purification can be achieved by any means known to those of skill in the art, and can include the extraction of total oil from an organism which produces DHA. In some embodiments, EPA, ARA, and/or DPAn6 are then removed from the total oil, for example, via chromatographic methods. Alternatively, DHA purification can be achieved by extraction of total oil from an organism which produces DHA, but produces little, if any, amount of EPA, ARA, DPAn6, and/or flavonoids. DHA purification can include the extraction of total oil from an organism which produces DHA. In some embodiments, EPA, ARA and/or DPAn6 are then removed from the total oil, for example, via chomatographic methods. In some embodiments, the oil can be diluted with sunflower oil to achieve the desired concentration of fatty acids.

Microbial oils useful in the present invention can be recovered from microbial sources by any suitable means known to those in the art. For example, the oils can be recovered by extraction with solvents such as chloroform, hexane, methylene chloride, methanol and the like, or by supercritical fluid extraction. Alternatively, the oils can be extracted using extraction techniques, such as are described in U.S. Pat. No. 6,750,048 and International Pub. No. WO 2001/053512 both filed Jan. 19, 2001, and entitled “Solventless extraction process,” both of which are incorporated herein by reference in their entirety. Processes for the preparation of various forms of DHA are also described in, among others, US Patent Publication No. 2009/0023808 “Production and Purification of Esters of Polyunsaturated Fatty Acids” by Raman et al., and US Patent Publication No. 2007/0032548 “Polyunsaturated fatty acids for treatment of dementia and pre-dementia-related condition” by Ellis, incorporated herein by reference.

Additional extraction and/or purification techniques are taught in International Pub. No. WO 2001/076715; International Pub. No. WO 2001/076385; U.S. Pub. No. 2007/0004678; U.S. Pub. No. 2005/012739; U.S. Pat. No. 6,399,803; and International Pub. No. WO 2001/051598; all of which are incorporated herein by reference in their entirety. The extracted oils can be evaporated under reduced pressure to produce a sample of concentrated oil material. Processes for the enzyme treatment of biomass for the recovery of lipids are disclosed in International Pub. No. WO 2003/09628; U.S. Pub. No. 2005/0170479; EP Pat. Pub. 0776356 and U.S. Pat. No. 5,928,696, the last two entitled “Process for extracting native products which are water-soluble from native substance mixtures by centrifugal force,” all of which are incorporated herein by reference in their entirety.

Microbial oils useful in the present invention can be recovered from microbial sources by any suitable means known to those in the art. For example, the oils can be recovered by extraction with solvents such as chloroform, hexane, methylene chloride, methanol and the like, or by supercritical fluid extraction. Alternatively, the oils can be extracted using extraction techniques, such as are described in U.S. Pat. No. 6,750,048 and International Pub. No. WO 2001/053512, both filed Jan. 19, 2001, and entitled “Solventless extraction process,” both of which are incorporated herein by reference in their entirety.

Additional extraction and/or purification techniques are taught in International Pub. No. WO 2001/076715; International Pub. No. WO 2001/076385; U.S. Pub. No. 2007/0004678; U.S. Pub. No. 2005/0129739; U.S. Pat. No. 6,399,803; and International Pub. No. WO 2001/051598; all of which are incorporated herein by reference in their entirety. The extracted oils can be evaporated under reduced pressure to produce a sample of concentrated oil material. Processes for the enzyme treatment of biomass for the recovery of lipids are disclosed in International Pub. No. WO 2003/092628; U.S. Pub. No. 2005/0170479; EP Pat. Pub. 0776356 and U.S. Pat. No. 5,928,696, the last two entitled “Process for extracting native products which are not water-soluble from native substance mixtures by centrifugal force,” all of which are incorporated herein by reference in their entirety.

In some embodiments, the DHA can be prepared as esters using a method comprising: a) reacting a composition comprising polyunsaturated fatty acids in the presence of an alcohol and a base to produce an ester of a polyunsaturated fatty acid from the triglycerides; and b) distilling the composition to recover a fraction comprising the ester of the polyunsaturated fatty acid, optionally wherein the method further comprises: c) combining the fraction comprising the ester of the polyunsaturated fatty acid with urea in a medium; d) cooling or concentrating the medium to form a urea-containing precipitate and a liquid fraction; and e) separating the precipitate from the liquid fraction. See, e.g., U.S. patent publication no. US2009/0023808, incorporated by reference herein in its entirety. In some embodiments, the purification process includes starting with refined, bleached, and deodorized oil (RBD oil), then performing low temperature fractionation using acetone to provide a concentrate. The concentrate can be obtained by base-catalyzed transesterification, distillation, and silica refining to produce the final DHA product.

Methods of determining purity levels of fatty acids are known in the art, and can include, e.g., chromatographic methods such as, e.g., HPLC silver ion chromatographic columns (ChromSpher 5 Lipids HPLC Column, Chrompack, Raritan N.J.). Alternatively, the purity level can be determined by gas chromatography, with or without converting DHA to the corresponding methyl ester.

In some embodiments, the DHA esters of the invention are derived from undiluted oil from a single cell microorganism, and in some embodiments, from undiluted DHASCO-T (Martek Biosciences Corporation, Columbia, Md.). In some embodiments, the oil from which DHA of the invention are derived include single cell microorganism oils that are manufactured by a controlled fermentation process followed by oil extraction and purification using methods common to the vegetable oil industry. In certain embodiments, the oil extraction and purification steps include refining, bleaching, and deodorizing. In some embodiments, the undiluted DHA oil comprises about 40% to about 50% DHA by weight (about 400-500 mg DHA/g oil). In certain embodiments, the undiluted DHA oil is enriched by cold fractionation (resulting in oil containing about 60% w/w of DHA triglyceride), which DHA fraction optionally can be transesterified, and subjected to further downstream processing to produce the active DHA of the invention. In some embodiments of the invention, downstream processing of the oil comprises distillation and/or silica refinement.

Thus, to produce oil form which DHA of the invention are derived, in certain aspects of the invention, the following steps are used: fermentation of a DHA producing microorganism; harvesting the biomass; spray drying the biomass; extracting oil from the biomass; refining the oil; bleaching the oil; chill filtering the oil; deodorizing the oil; and adding an antioxidant to the oil. In some embodiments, the microorganism culture is progressively transferred from smaller scale fermenters to a production size fermenter. In some embodiments, following a controlled growth over a pre-established period, the culture is harvested by centrifugation then pasteurized and spray dried. In certain embodiments, the dried biomass is flushed with nitrogen and packaged before being stored frozen at −20° C. In certain embodiments, the DHA oil is extracted from the dried biomass by mixing the biomass with n-hexane or isohexane in a batch process which disrupts the cells and allows the oil and cellular debris to be separated. In certain embodiments, the solvent is then removed.

In some embodiments, the crude DHA oil then undergoes a refining process to remove free fatty acids and phospholipids. The refined DHA oil is transferred to a vacuum bleaching vessel to assist in removing any remaining polar compounds and pro-oxidant metals, and to break down lipid oxidation products. The refined and bleached DHA oil undergoes a final clarification step by chilling and filtering the oil to facilitate the removal of any remaining insoluble fats, waxes, and solids.

Optionally, the DHA is deodorized under vacuum in a packed column, counter current steam stripping deodorizer. Antioxidants such as ascorbyl palmitate and alpha-tocopherol can optionally be added to the deodorized oil to help stabilize the oil. In some embodiments, the final, undiluted DHA oil is maintained frozen at −20° C. until further processing.

In some embodiments, the DHA oil is converted to DHA ester by methods known in the art. In some embodiments, DHA esters of the invention are produced from DHA oil by the following steps: cold fractionation and filtration of the DHA oil (to yield for example about 60% triglyceride oil); direct transesterification (to yield about 60% DHA ethyl ester); molecular distillation (to yield about 88% DHA ethyl ester); silica refinement (to yield about 90% DHA ethyl ester); and addition of an antioxidant.

In some embodiments, the cold fractionation step is carried out as follows: undiluted DHA oil (triglyceride) at about 500 mg/g DHA is mixed with acetone and cooled at a controlled rate in a tank with −80° C. chilling capabilities. Saturated triglycerides crystallize out of solution, while polyunsaturated triglycerides at about 600 mg/g DMA remain in the liquid state. The solids containing about 300 mg/g are filtered out with a 20 micron stainless steel screen from the liquid stream containing about 600 mg/g DHA. The solids stream is then heated (melted) and collected. The 600 mg/g DMA liquid stream is desolventized with heat and vacuum and then transferred to the transesterification reactor.

In some embodiments, the transesterification step is carried out on the 600 mg/g DHA oil, wherein the transesterification is done via direct transesterification using ethanol and sodium ethoxide. The transesterified material DHA ethyl ester (“DHA-EE”) is then subject to molecular distillation and thus, further distilled (3 passes, heavies, lights, heavies) to remove most of the other saturated fatty acids and some sterols and non-saponifiable material. The DHA-EE is further refined by passing it through a silica column.

DHA free fatty acids can be made using, for example, the DHA containing oils described above. In some embodiments, the DHA-FFA can be obtained from DHA esters. DHA triglycerides, for example, can be saponified followed by a urea adduction step to make free fatty acids.

Additional fatty acids can be present in the dosage form or unit dose. These fatty acids can include fatty acids that were not removed during the purification process, i.e., fatty acids that were co-isolated with DHA from an organism. These fatty acids can be present in various concentrations. In some embodiments, the dosage form comprises 0.1% to 60% of one or more of the following fatty acids, or esters thereof: (a) capric acid; (b) lauric acid; (c) myristic acid; (d) palmitic acid, (e) palmitoleic acid; (f) stearic acid; (g) oleic acid; (h) linoleic acid; (i) α-linolenic acid; (j) docosapentaenoic acid 22:5n-3, 22:5w3 (DPAn3); and (k) 4,7,10,13,16,19,22,25 octacosaoctaenoic acid (C28:8). In some embodiments, the dosage form comprises 20% to 40% of one or more of the following fatty acids, or esters thereof: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); and (k) 4,7,10,13,16,19,22,25 octacosaoctaenoic acid (C28:8). In some embodiments, the dosage form comprises less than about 1% each of the following fatty acids, or esters thereof: (a) capric acid; (b) lauric acid; (c) myristic acid; (d) palmitic acid, (e) palmitoleic acid; (f) stearic acid; (g) oleic acid; (h) linoleic acid; (i) α-linolenic acid; (j) docosapentaenoic acid 22:5n-3, 22:5w3 (DPAn3); and (k) 4,7,10,13,16,19,22,25 octacosaoctaenoic acid (C28:8).

In some embodiments, the dosage form or unit dose is characterized by a fatty acid content of about 0.1% to about 20% (w/w) of one or more of the following fatty acids or esters thereof: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); and (k) 4,7,10,13,16,19,22,25 octacosaoctaenoic acid (C28:8).

In some embodiments, a dosage form or unit dose is characterized by a fatty acid content of about 1.0% to about 5% (w/w) of one or more of the following fatty acids or esters thereof: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); and (k) 4,7,10,13,16,19,22,25 octacosaoctaenoic acid (C28:8).

In some embodiments, a dosage form or unit dose is characterized by a fatty acid content of less than about 1% (w/w) each of the following fatty acids or esters thereof: (a) capric acid; (b) lauric acid; (c) myristic acid; (d) palmitic acid; (e) palmitoleic acid; (f) stearic acid; (g) oleic acid; (h) linoleic acid; (i) α-linolenic acid; (j) docosapentaenoic acid 22:5n-3, 22:5w3 (DPAn3); (k) docosapentaenoic acid 22:5n-6 (DPAn6); and (l) 4,7,10,13,16,19,22,25 octacosaoctaenoic acid (C28:8). In some embodiments, the dosage form of the present invention does not contain a detectable amount of docosapentaenoic acid 22:5n-3, 22:5w3 (DPAn3); docosapentaenoic acid 22:5n-6 (DPAn6); and/or 4,7,10,13,16,19,22,25 octacosaoctaenoic acid (C28:8).

In some of embodiments of DHA dosage form described herein, the dosage form or the unit dose is characterized by one or more the following fatty acids (or esters thereof), expressed as wt % of the total fatty acid content. The embodiments provided herein may further comprise about 2% or less (w/w) of capric acid (C10:0). The embodiments herein may further comprise about 6% or less of lauric acid (C12:0). The embodiments herein may further comprise about 20% or less, or about 5% to about 20% (w/w) of myristic acid (C14:0). The embodiments herein may further comprise about 20% or less, or about 5% to about 20% (w/w) of palmitic acid (C16:0). The embodiments herein may further comprise about 3% or less of palmitoleic acid (C16:1n-7). The embodiments herein may further comprise about 2% or less of stearic acid (C18:0). The embodiments herein may further comprise about 40% or less, or about 10% to about 40% of oleic acid (C18:1n-9). The embodiments herein may further comprise about 5% or less of linoleic acid (C18:2). The embodiments herein may further comprise about 2% or less of nervonic acid (C24:1). The embodiments herein may further comprise about 3% or less of other fatty acids or esters thereof. The DHA dosage form or unit dose with the preceding characteristics may comprise DHASCO® (Martek Biosciences, Columbia, Md.), an oil derived from Crypthecodinium cohnii containing docosahexaenoic acid (DHA).

An exemplary DHA (triglyceride) containing oil derived from Crypthecodinium cohnii is characterized by the specified amount of components listed in Table 1, where “Max” refers to the amount of the component that can be present up to the specified amount.

TABLE 1

| | Concentration (wt/wt) |
|---|---|
| Fatty Acids | |
| 10:0 | Max 2% |
| 12:0 | Max 6% |
| 14:0 | 5%-20% |
| 16:0 | 5%-20% |
| 16:1 | Max 3% |
| 18:0 | Max 2% |
| 18:1 | 10%-40% |
| 18:2 | Max 5% |
| 22:6 DHA | 40% to 45% |
| 24:1 | Max 2% |
| Others | Max 3% |
| Elemental Composition | |
| Arsenic | Max 0.5 ppm |
| Copper | Max 0.1 ppm |
| Iron | Max 0.5 ppm |
| Lead | Max 0.2 ppm |
| Mercury | Max 0.04 ppm |
| Phosphorous | Max 10 ppm |
| Chemical Characteristics | |
| Peroxide value | Max 5 meq/kg |
| Free fatty acid | Max 0.4% |
| Unsaponifiable Matter | Max 3.5% |

An exemplary undiluted DHA (triglyceride) containing oil derived from Crypthecodinium cohnii is characterized by an amount of DHA described herein, and one or more, or all of the features listed below in Table 2, where “Max” refers to the amount of the component that can be present up to the specified amount.

TABLE 2
Characteristics of Undiluted DHA Oil

| Test | Specification |
|---|---|
| DHA content mg/DHA/g oil | Min 480 mg/g |
| Free Fatty Acid | Max 0.4% |
| Peroxide Value (PV) | Max 5 meq/kg |
| Anisidine Value (AV) | Max 20 |
| Moisture and Volatiles (M & V) | Max 0.02% |
| Unsaponifiable Matter | Max 3.5% |
| Insoluble Impurities | Max 0.1% |
| Trans Fatty Acid | Max 1% |
| Arsenic | Max 0.5 ppm |
| Cadmium | Max 0.2 ppm |
| Chromium | Max 0.2 ppm |
| Copper | Max 0.1 ppm |
| Iron | Max 0.5 ppm |
| Lead | Max 0.2 ppm |
| Manganese | Max 0.04 ppm |
| Mercury | Max 0.04 ppm |
| Molybdenum | Max 0.2 ppm |
| Nickel | Max 0.2 ppm |
| Phosphorus | Max 10 ppm |
| Silicon | Max 500 ppm |
| Sulfur | Max 100 ppm |
| 18:1 n-9 Oleic Acid | Max 10% |
| 20:5 n-3 EPA | Max 0.1% |
| Unknown Fatty Acids | Max 3% |

In some embodiments of the DHA dosage forms described herein, the dosage form is characterized by one or more the following fatty acids (or esters thereof), expressed as wt % of the total fatty acid content. The embodiments provided herein can further comprise about 2% or less of capric acid (C10:0). The embodiments provided herein can further comprise about 6% or less of lauric acid (C12:0). The embodiments provided herein can further comprise about 20% or less, or about 10% to about 20% of myristic acid (C14:0). The embodiments provided herein can further comprise about 15% or less, or about 5% to about 15% (w/w) of palmitic acid (C16:0). The embodiments provided herein can further comprise about 5% or less of palmitoleic acid (C16:1n-7). The embodiments provided herein can further comprise about 2% or less of stearic acid (C18:0). The embodiments provided herein can further comprise about 20% or less, or about 5% to about 20% of oleic acid (C18:1n-9). The embodiments provided herein can further comprise about 2% or less of linoleic acid (C18:2). The embodiments provided herein can further comprise about 2% or less of nervonic acid (C24:1). The embodiments provided herein can further comprise about 3% or less of other fatty acids. The DHA dosage form with the preceding characteristics can be an oil derived from Crypthecodinium cohnii containing docosahexaenoic acid (DHA).

In some embodiments, the dosage form comprises, measured in percentage of free fatty acid, about 35-65%, 40-55%, 35-57%, or 57-65% DHA (22:6 n-3); about 0-2% capric acid (10:0); about 0-6% lauric acid (12:0); about 10-20% myristic acid (14:0); about 5-15% palmitic acid (16:0); about 0-5% palmitoleic acid (16:1); about 0-2% stearic acid (18:0); about 5-20% or 5-25% oleic acid (18:1); about 0-2% linoleic acid (18:2); and about 0-2% nervonic acid (24:1, n-9). In one embodiment, such an oil is from a microorganism of the genus Thraustochytrium. In another embodiment, the free fatty acid content is less than 0.4%

The present invention can also provide compositions comprising at least about 40 wt % DHA and at least about 0.1 wt of DPA (n-3). In some embodiments, the compositions comprise at least about 55, 56, 57, 58, 59, 60, 61, 62, 63, 64, 65 wt. % DHA, optionally in triglyceride form, as a percentage of total fatty acids.

An exemplary DHA-containing oil derived from Crypthecodinium cohnii is characterized by the specified amount of components listed in Table 3, where “Max” refers to the amount of the component that can be present up to the specified amount.

TABLE 3

| | Concentration (wt/wt) |
|---|---|
| Fatty Acids | |
| 10:0 | 0-2% |
| 12:0 | 0-6% |
| 14:0 | 10%-20% |
| 16:0 | 5%-15% |
| 16:1 | 0-5% |
| 18:0 | 0-2% |
| 18:1 | 5%-20% |
| 18:2 | 0-2% |
| 22:6 n-3 DHA | 57%-65% |
| 24:1 | 0-2% |
| Others | 0-3% |
| Elemental Composition | |
| Arsenic | Max 0.5 ppm |
| Copper | Max 0.1 ppm |
| Iron | Max 0.5 ppm |
| Lead | Max 0.2 ppm |
| Mercury | Max 0.2 ppm |
| Phosphorous | Max 10 ppm |
| Chemical Characteristics | |
| Peroxide value | Max 5 meq/kg |
| Free fatty acid | Max 0.4% |
| Unsaponifiable Matter | Max 3.5% |
| Trans fatty acids | <3.5% |
| Moisture and Volatiles | <0.1% |
| Insoluble impurities | <0.1% |

In some embodiments of the DHA dosage forms described herein, the dosage form or unit dose is characterized by one or more the following fatty acids (or esters thereof), expressed as wt % of the total fatty acid content: The embodiments provided herein can further comprise about 0.1% or less of myristic acid (C14:0) or is not detectable. The embodiments provided herein can further comprise about 0.5% or less of palmitic acid (C16:0). The embodiments provided herein can further comprise about 0.5% or less of palmitoleic acid (C16:1n-7). The embodiments provided herein can further comprise about 0.5% or less of stearic acid (C18:0), or is not detectable. The embodiments provided herein can further comprise about 4% or less of oleic acid (C18:1n-9). The embodiments provided herein can further comprise less than 0.1% of linoleic acid (C18:2) or is not detectable. The embodiments provided herein can further comprise less than 0.1% of eicospentaenoic acid (C20:5) or is not detectable. The embodiments provided herein can further comprise about 2% or less of decosapentaenoic acid (22:5n-3). The embodiments provided herein can further comprise about 1% or less of octacosaoctaenoic acid (29:8 n-3). The embodiments provided herein can further comprise about 0.5% or less of tetracosaenoic acid (24:1ω9). The embodiments provided herein can further comprise about 1% or less of other fatty acids. The DHA in dosage form or unit dose with the preceding characteristics can be in the form of a DHA ester, preferably an alkyl ester, such as a methyl ester, ethyl ester, propyl ester, or combinations thereof, prepared from an algal oil prepared from the Thraustochytrid, Schizochytrium sp.

In some embodiments, the DHA composition may comprise DHASCO®. DHASCO® is an oil derived from Crypthecodinium cohnii containing high amounts of docosahexaenoic acid (DHA), and more specifically contains the following approximate exemplary amounts of these fatty acids, as a percentage of the total fatty acids: myristic acid (14:0) 10-20%; palmitic acid (16:0) 10-20%; palmitoleic acid (16:1) 0-2%; stearic acid (18:0) 0-2%; oleic acid (18:1) 10-30%; linoleic acid (18:2) 0-5%; arachidic acid (20:0) 0-1%; behenic acid (22:0) 0-1%; docosapentaenoic acid (22:5) 0-1%; docosahexaenoic acid (22:6) (DHA) 40-45%; nervonic acid (24:1) 0-2%; and others 0-3%.

The present invention also provides compositions comprising at least about 40 wt. % DHA and at least about 0.1 wt. % of 4,7,10,13,16,19,22,25 octacosaoctaenoic acid (C28:8). In some embodiments, the compositions comprise at least about 55, 56, 57, 58, 59, 60, 61, 62, 63, 64, 65 wt. % DHA, optionally in triglyceride form, as a percentage of total fatty acids. In other embodiments, the compositions comprise at least about 90, 91, 92, 93, 94, 95, 96, 97, 98, or 99 wt. % of DHA, optionally in ethyl ester form, as a percentage of total fatty acids. In certain embodiments, the amount of C28:8 in the compositions may be at least about 0.2, 0.3, 0.4, 0.5, 0.6, 0.7, 0.8, 0.9, 1.0, 1.1, 1.2, 103, 1.4 or 1.5 wt. %. The C28:8 may be present in any form, including triglyceride or ester form. For example, the C28:8 may be present in ethyl ester form.

In other embodiments, the compositions comprise at least about 90, 91, 92, 93, 94, 95, 96, 97, 98, or 99 wt. % of DHA, optionally in ethyl ester form, as a percentage of total fatty acids. In certain embodiments, the amount of DPA (n-3) in the compositions may be at least about 0.1, 0.2, 0.3, 0.4, 0.5, 0.6, 0.7, 0.8, 0.9, or 1.0 wt. % of DPA (n-3). The DPA (n-3) may be present in triglyceride or ester form. For example, the DPA (n-3) may be present in ethyl ester form. In certain embodiments, the compositions comprise all three of the DHA, C28:8 and DPA (n-3) in the concentration ranges specified above.

In further embodiments, the compositions may comprise less than about 1.0, 0.9, 0.8, 0.7, 0.6, 0.5, 0.4, 0.3, 0.2, or 0.1 wt′ % EPA in addition to the DHA and C28:8. In one embodiment, the compositions may comprise less than about 0.25 wt. % EPA. The EPA may be present in any form, including triglyceride or ester form. In some embodiments, the compositions may comprise 0 wt. % EPA.

The present invention also provides compositions comprising at least about 90 wt. % of DHA and at least one additional fatty acid or a derivative thereof. In some embodiments, the amount of DHA in the compositions may be at least about 91, 92, 93, 94, 95, 96, 97, 98, or 99 wt. %. In certain embodiments, the additional fatty acid may have a boiling point of about 150-170° C. at a pressure of 0.8 mm Hg.

An exemplary DHA-containing oil derived from the algal oil of Crypthecodinium cohnii, wherein the DHA comprises an ethyl ester, can be characterized by the specified amount of components listed in Table 4, where “Max” refers to the amount of the component that can be present up to the specified amount.

TABLE 4

| | |
|---|---|
| DHA content (mg/g) | 855-945 |
| Fatty Acid Content: % of total EE | |
| Eicosapentaenoic Acid (20:5ω3) | ND |
| Myristic Acid (14:0) | 0.1% |
| Palmitic Acid (16:0) | 0.5% |
| Palmitoleic Acid (16:1ω7) | 0.4% |
| Stearic Acid (18:0) | ND |
| Oleic Acid (18:1ω9) | 4% |
| Linoleic Acid (18:2ω6) | ND |
| Docosapentaenoic acid (22:5ω3) | 1.3% |
| Octacosaoctaenoic acid (28:8ω3) | 0.9% |
| Tetracosaenoic Acid (24:1ω9) | 0.3% |
| Others | 1.1% |
| Elemental Composition | |
| Arsenic | Max 0.5 ppm |
| Copper | Max 0.1 ppm |
| Iron | Max 0.5 ppm |
| Lead | Max 0.2 ppm |
| Mercury | Max 0.04 ppm |
| Chemical Characteristics | |
| Peroxide value | Max 10.0 meq/kg |

ND = not detectable

In some embodiments of the DHA dosage form described herein, the dosage form or unit dose is characterized by one or more the following fatty acids (or esters thereof), expressed as wt % of the total fatty acid content. The embodiments provided herein may further comprise about 12% or less, or about 6% to about 12% of myristic acid (C14:0). The embodiments provided herein may further comprise about 28% or less, or about 18 to about 28% of palmitic acid (C16:0). The embodiments provided herein may further comprise about 2% or less of stearic acid (C18:0). The embodiments provided herein may further comprise about 8% or less of oleic acid (C18:1n-9). The embodiments provided herein may further comprise about 2% or less of linoleic acid (C18:2). The embodiments provided herein may can further comprise about 2% or less of arachidonic acid (C20:4). The embodiments provided herein may further comprise about 3% or less of eicospentaenoic acid (C20:5). The embodiments provided herein may further comprise about 18% or less, or about 12% to about 18% of decosapentaenoic acid (22:5n-6). The embodiments provided herein may further comprise about 10% or less of other fatty acids. In some of these embodiments, the ratio of wt % of DHA to wt % of DPAn6 is about 2.5 to about 2.7. The DHA dosage form or unit dose with the preceding characteristics may comprise Life's DHA™ (also formerly referenced as DHA-S and DHASCO-S, Martek Biosciences, Columbia, Md.), an oil derived from the Thraustochytrid, Schizochytrium sp., that contains a high amount of DHA and also contains docosapentaenoic acid (n-6) (DPAn-6).

In some embodiments, more specifically, DHA-S (Martek Biosciences, Columbia, Md.) contains the following approximate exemplary amounts of these fatty acids, as a percentage of total fatty acids: myristic acid (14:0) 8.71%; palmitic acid (16:0) 22.15%; stearic acid (18:0) 0.66%; linoleic acid (18:2) 0.46%; arachidonic acid (20:4) 0.52%; eicosapentenoic acid (20:5, n-3) 1.36%; docosapentaenoic acid (22:5, n-6) (DPAn-6) 16.28%; docosahexaenoic acid (DHA) (22:6, n-3) 41.14%; and others 8%.

In some embodiments, the dosage form comprises, measured in percentage of free fatty acid, about 35-45% DHA (22:6 n-3); about 0-2% lauric acid (12:0); about 5-10% myristic acid (14:0); about 5-20% palmitic acid (16:0); about 0-5% palmitoleic acid (16:1); about 0-5% stearic acid (18:0); about 0-5% vaccenic acid or oleic acid (18: 1 n-7 and n-9, respectively); about 0-2% linoleic acid (18:2, n-6); about 0-5% stearidonic acid (18:4 n-3); about 0-10% 20:4 n-3, n-5, or n-6; about 0-2% adrenic acid 22:4 n-6; about 0-5% DPA n-3 (22:5); about 10-25% DPA n-6 (22:5); and 0-2% 24:0. In one embodiment, such an oil is from a microorganism of the genus Schizochytrium.

An exemplary DHA containing oil derived from Schizochytrium sp. is characterized by the specified amount of components listed in Table 5, where “Max” refers to the amount of the component that can be present up to the specified amount.

TABLE 5

| | Concentration (wt/wt) |
|---|---|
| Fatty Acids | |
| 14:0 | 6%-12% |
| 16:0 | 18%-28% |
| 18:0 | Max 2% |
| 18:1 | Max 8% |
| 18:2 | Max 2% |
| 20:4 ARA | Max 2% |
| 20:5 EPA | Max 3% |
| 22:5n-6 DPA | 12%-18% |
| 22:6 DHA | Min. 35% |
| Others | Max 10% |
| Elemental Composition | |
| Arsenic | Max 0.2 ppm |
| Copper | Max 0.05 ppm |
| Iron | Max 0.2 ppm |
| Lead | Max 0.1 ppm |
| Mercury | Max 0.04 ppm |
| Chemical Characteristics | |
| Peroxide value | Max 5 meq/kg |
| Free fatty acid | Max 0.25% |
| Moisture and Volatiles | Max 0.05% |
| Unsaponifiable Matter | Max 4.5% |
| Trans fatty acids | Max 1% |

Compositions useful in the methods herein also include compositions that comprise at least about 90 wt. % of a combination of DPA (n-6) and DHA. In certain embodiments, the compositions may comprise at least about 91, 92, 93, 94, 95, 96, 97, 98, or 99 wt. % of a combination of DPA (n-6) and DHA. In some embodiments, the compositions may comprise at least about 10 wt. % DHA and at least about 10 wt. % DPA (n-6). In other embodiments, the compositions may comprise at least about 15 or 20 wt. % DHA and at least about 15 or 20 wt. % DPA (n-6).

The present invention also provides compositions comprising at least about 90 wt. % of a combination of DPA (n-6) and DHA, and at least one additional fatty acid or a derivative, such as an ester, thereof. In certain embodiments, the compositions may comprise at least about 91, 92, 93, 94, 95, 96, 97, 98, or 99 wt. % of a combination of DPA (n-6) and DHA. In some embodiments, the additional fatty acid may have a boiling point of about 150-170° C. at a pressure of 0.8 mm Hg.

The DHA/DPA (n-6) compositions described above may further comprise less than about 4% of a saturated fatty acid or an ester thereof. In certain embodiments, the compositions may comprise less than about 3.5%, 3.0%, 2.5%, 2.0%, 1.5%, 1.0% or 0.5% of a saturated fatty acid or a derivative thereof.

The DHA in an oil may be in the form of a DHA ester, preferably an alkyl ester, such as a methyl ester, ethyl ester, propyl ester, or combinations thereof, prepared from an algal oil prepared from derived from the Thraustochytrid, Schizochytrium sp. An exemplary DHA (ethyl esters) containing oil derived from Schizochytrium sp. is characterized by the specified amount of components listed in Table 4 of WO 2009/006317, incorporated by reference herein. In some of these embodiments, an oil comprises DHA≧than about 57% (w/w), particularly ≧about 70% (w/w) of the total fatty acid content of the oil or unit dose.

In some embodiments, the composition or oil is characterized by one or more the following fatty acids (or esters thereof, particularly ethyl esters), expressed as wt % of the total fatty acid content. The embodiments provided herein may further comprise about 0.5% or less (w/w) of lauric acid (C12:0). The embodiments provided herein may further comprise about 2% or less (w/w) of myristic acid (C14:0). The embodiments provided herein may further comprise about 0.5% or less (w/w) of myristoleic acid (C14: 1). The embodiments provided herein may further comprise about 1% or less of palmitic acid (C16:0). The embodiments provided herein may further comprise about 1% or less (w/w) of linoleic acid (C18:2) (n-6). The embodiments provided herein may further comprise about 3% or less (w/w) of dihomo gamma linolenic acid (C20:3) (n-6). The embodiments provided herein may further comprise about 0.5% or less (w/w) of eicosatrienoic (C20:3) (n-3). The embodiments provided herein may further comprise about 1% or less (w/w) of arachidonic acid (C20:4). The embodiments provided herein may further comprise about 3% or less (w/w) of eicosapentaenoic acid (C20:5) (n-3). The embodiments provided herein may further comprise about 3% or less (w/w) of docosatrienoic acid (22:3). The embodiments provided herein may further comprise about 27% or less (w/w) of decosapentaenoic acid (22:5) (n-6). The embodiments provided herein may further comprise about 10% or less (w/w) of other components. In some of these embodiments, the ratio of wt % of DHA to wt % of DPAn6 is about 2.5 to about 2.7. An oil with the preceding characteristics may comprise ethyl ester oil derived from the oil of Thraustochytrid, Schizochytrium sp.

An exemplary DHA (free fatty acid) containing oil is characterized by the specified amount of components listed in Table 6:

TABLE 6

| | Concentration (wt/wt) |
|---|---|
| Fatty Acids | |
| 10:0 | Max 0.5% |
| 12:0 | Max 0.5% |
| 14:0 | Max 0.5% |
| 14:1 | Max 0.5% |
| 16:0 | Max 0.5% |
| 16:1 | Max 0.5% |
| 18:1 (n-9) | Max 0.5% |
| 20:5 (n-3) EPA | Max 0.5% |
| 22:5 (n-3) DPA | Max 1% |
| 22:6 (n-3) DHA | Min 95% |
| 28:8 | Max 1.5% |
| Chemical Characteristics | |
| Docosahexaenoic acid | 946 mg/g |
| Docosahexaenoic acid | 98% |
| Free Fatty Acids | 93% |
| Trans Fatty Acids | <1% |

In some embodiments, the present invention further includes use of compositions comprising at least about 70 wt. % DHA and at least about 15, 20, or 25 wt. % DPA (n-6).

In some embodiments, the saturated fatty acid or an ester thereof may contain less than 20 carbons, such as, for example, a saturated fatty acid or an ester thereof that contains 19, 18, 17. 16, 15, 14, 13, 12, 11, 10, 9 or 8 carbons. In certain embodiments, the saturated fatty acid or ester thereof may contain 14 or 16 carbons.

In some embodiments, the composition of DHA may further comprise vitamin E. Compounds of the vitamin E group are fat-soluble vitamins with antioxidant properties and include eight related α-, β-, γ-, and δ-tocopherols and the corresponding four tocotrienols. In some embodiments, the vitamin E in the composition is a tocopherol. In some embodiments, the tocopherol is selected from α-, β-, γ-, and δ-tocopherols, or combinations thereof.

For treating the subject with arthritis, various dosage amounts of DHA can be administered to a subject. The term “daily dosage,” “daily dosage level,” “daily dosage amount” or “dose per day” refers to the total amount of DHA administered per day (about 24 hour period). Thus, for example, administration of DHA to a subject at a daily dosage of about 2 g means that the subject receives a total of about 2 g of DHA in a day or on a daily basis, whether the DHA is administered as a single dosage form comprising about 2 g DHA, or alternatively, four dosage forms comprising about 500 mg DHA each (for a total of about 2 g DHA). In some embodiments, the daily amount or dose per day of DHA is about 12.4 g DHA or less, about 430 mg to about 12.4 g DHA, about 500 mg to about 3.7 g of DHA, about 750 mg to about 3.5 g DHA, or about 1 g to about 2 g DHA. In some embodiments, the daily amount of DHA is about 520 mg to about 4 g, about 540 mg to about 4 g, about 560 mg to about 4 g, or about 580 mg to 4 g. In some embodiments, the daily amount of DHA is less than about 3.8 g DHA, about 900 mg to about 3.6 g DHA, or about 1.8 g to about 2.7 g of DHA. In some embodiments, the daily amount of DHA comprises about 430 mg, about 450 mg, about 500 mg, about 550 mg, about 600 mg, about 650 mg, about 700 mg, about 750 mg, about 800 mg, about 850 mg, about 900 mg, about 950 mg, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 10.5 g, about 11 g, about 11.5 g, about 12 g, or about 12.4 g DHA.

In some embodiments, the daily amount or dose per day of DHA is about 0.8 g to about 4 g of DHA. In some embodiments, the dose per day of DHA is about 0.84 g to about 4 g of DHA. In some embodiments, the dose per day of DHA is about 0.84 g to about 1.5 g of DHA. In some embodiments, the dose per day of DHA is about 0.84 mg to about 1.0 g of DHA.

In some embodiments, the DHA is administered in a single dosage form, i.e., a dosage form, or in two or more dosage forms. As used herein, “dosage form” refers to the physical form of the route of administration. The term “dosage form” can refer to any traditionally used or medically accepted administrative forms, such as oral administrative forms, intravenous administrative forms, or intraperitoneal administrative forms. As used herein, a “dosage form” can also refer to an amount of DHA administered to a subject in a single dosage form, e.g., in a gelatin capsule. Alternatively, the term ‘dosage form’ refers to an amount of DHA that is administered to a subject at a single time period, e.g., two pills swallowed at the same time, or one pill swallowed shortly after another pill. The term “dosage form” can also refer to a unit of a solid, liquid, syrup, beverage, or food item, that is swallowed within a short period of time, e.g., within 1 minute, 2 minutes, 3 minutes, 5 minutes, 10 minutes, 20 minutes, or 30 minutes. In some embodiments, as used herein, the term “dosage form” can also refer unformulated oils.

In some embodiments, the DHA is administered in a single dosage form, i.e., a unit dose. As used herein, a “unit dose” refers to an amount of DHA administered to a subject in a single dosage form, e.g., in a gel capsule. The term “unit dose” can also refer to a unit of pharmaceutically suitable solid, liquid, syrup, beverage, or food item, that is swallowed within a short period of time, e.g., within 1 minute, 2 minutes, 3 minutes, 5 minutes, 10 minutes, 20 minutes, or 30 minutes.

In some embodiments, the subject to be treated can be administered at least one unit dose per day. In some embodiments, the dosage forms can be taken in a single application or multiple applications per day. For example, if four capsules are taken daily, each capsule comprising about 500 mg DHA, then all four capsules could be taken once daily, or 2 capsules could be taken twice daily, or 1 capsule could be taken every 6 hours. Various amounts of DHA can be in a unit dose. In some embodiments, the dosage form comprises less than about 12.4 g of DHA, less than about 10 g of DHA, about 430 mg to about 4.8 g DHA, about 450 mg to about 4.6 g of DHA, or about 500 mg to about 4 g DHA. In some embodiments, the unit dose comprises about 430 mg to about 12.4 g DHA. In some embodiments, the unit dose comprises about 430 mg, about 450 mg, about 500 mg, about 550 mg, about 600 mg, about 650 mg, about 700 mg, about 750 mg, about 800 mg, about 850 mg, about 900 mg, about 950 mg, about 1 g, about 1.5 g, about 2 g, about 2.5 g, about 3 g, about 3.5 g, about 4 g, about 4.5 g, about 5 g, about 5.5 g, about 6 g, about 6.5 g, about 7 g, about 7.5 g, about 8 g, about 8.5 g, about 9 g, about 9.5 g, about 10 g, about 10.5 g, about 11 g, about 11.5 g, about 12 g, or about 12.4 g DHA.

In some embodiments, where the DHA is in the form of an ester, preferably an alkyl ester, such as a methyl ester, ethyl ester, propyl ester, or combinations thereof, the dosage form or unit dose can comprise about 430 mg to about 480 mg of DHA ester, particularly the ethyl ester. In some embodiments, the dosage form or the unit dose can comprise about 860 mg to about 950 mg, or about 870 mg to about 930 mg of the DI-IA ester, particularly the ethyl ester.

In some embodiments, the daily dose is provided as a unit dose.

In some embodiments, the dosage form has a total weight of about 0.2 g to about 2 g. By way of example and not limitation, a capsule can contain a total weight an algal oil of about 0.2 g, where the algal oil contain comprises DHA at a certain wt % of the total fatty acid content of the algal oil. In some embodiments, the dosage form has a total weight of about 0.2 g, about 0.25, about 0.3 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, about 1 g or about 1.05 g.

In some embodiments, the DHA is about 30% (w/w) or more of the total fatty acid content of the dosage form or unit dose, about 30% to about 99.9% (w/w) of the total fatty acid content of the dosage form or unit dose, about 35% to about 99.9% (w/w) of the total fatty acid content of the dosage form or unit dose, about 35% to about 60% (w/w) of the total fatty acid content of the dosage form or unit dose, about 35% to about 50% (w/w) of the total fatty acid content of the dosage form or unit dose, about 37% to about 45% (w/w) of the total fatty acid content of the dosage form or unit dose, or about 38% to about 43% (w/w) of the total fatty acid content of the dosage form or unit dose. In some embodiments, the DHA is great than about 35%, about 37%, about 38%, about 39% or about 40% (w/w) of the total fatty acid content of the dosage form or unit dose. In some embodiments, the DHA is about 30% to about 99.5% (w/w) of the total fatty acid content of the dosage form or unit dose, or about 40% to about 65% (w/w) of the total fatty acid content of the dosage form or unit dose.

In some of these embodiments, the DHA comprises about 40% to about 45% (w/w) of the total fatty acid content of the dosage form or unit dose. In some of these embodiments, the DHA comprises about 35% to about 45% (w/w) of the total fatty acid content of the dosage form or unit dose. In some of embodiments, the DHA comprises about 55% to about 67% (w/w) of the total fatty acid content of the dosage form or unit dose. In some embodiments, the DHA comprises greater than about 70% (w/w) of the total fatty acid content of the dosage form or unit dose. In some embodiments, the DHA comprises greater than about 85% (wt/wt) of the total fatty acid content of the dosage form or unit dose. In some embodiments, the DHA comprises about 85,% to about 99.5% (w/w) of the total fatty acid content of the dosage form or unit dose.

In some embodiments, the DHA is greater than about 80% (w/w) of the total fatty acid content of the dosage form or unit dose, about 80% to about 99.9% (w/w) of the total fatty acid content of the dosage form or unit dose, about 85% to about 99% (w/w) of the total fatty acid content of the dosage form or unit dose, about 87% to about 98% (w/w) of the total fatty acid content of the dosage form or unit dose, or about 90% to about 97% (w/w) of the total fatty acid content of the dosage form or unit dose. In some embodiments, the DHA is great than about 95%, about 97%, about 98%, about 99% or about 99.5% (w/w) of the total fatty acid content of the dosage form or unit dose. In some embodiments, the DHA is greater than about 85% (w/w) of the total fatty acid content of the dosage form or unit dose

In some embodiments, the DHA comprises about 35% to about 96% of the weight of the dosage form or unit dose. In some embodiments, the DHA comprises about 38% to about 42% of the weight of the dosage form or unit dose. In some embodiments, the DHA in the dosage form or unit dose comprises about 35% to about 45% (w/w) of the total weight of the dosage form or unit dose. In some embodiments, the DHA in the dosage form or unit dose comprises about 55% to about 57% (w/w) of the total weight of the dosage form or unit dose. In some embodiments, the DHA in the dosage form comprises about 55% (w/w) to about 67% (w/w) of the total weight of the dosage form. In some embodiments, the DHA in the dosage form or unit dose comprises about 85% to about 96% (w/w) of the total weight of the dosage form or unit dose.

In some of these embodiments, the DHA in the dosage form or unit dose comprises greater than about 57% (w/w), particularly greater than about 70% (w/w) of the total fatty acid content of the dosage form or unit dose. The DHA in dosage form or unit dose with the preceding characteristics may be in the form of an ester, preferably an alkyl ester, such as a methyl ester, ethyl ester, propyl ester, or combinations thereof, prepared from an algal oil prepared from derived from the Thraustochytrid, Schizochytrium sp.

In some embodiments, the dosage form is a pharmaceutical dosage form. “Pharmaceutically acceptable” refers to compositions that are, within the scope of sound medical judgment, suitable for contact with the tissues of human beings and animals without excessive toxicity or other complications commensurate with a reasonable benefit/risk ratio. In some embodiments, the compounds (e.g., DHA), compositions, and dosage forms of the present invention are pharmaceutically acceptable.

In some embodiments, the dosage form is a nutraceutical dosage form. The term “nutraceutical” refers to any substance that is (1) a sole item of a meal or diet that provides medical and/or health benefits, or (2) a product that is intended to supplement the diet that bears or contains one or more of the following dietary ingredients: a vitamin, a mineral, an herb or other botanical, an amino acid, a dietary substance for use by man to supplement the diet by increasing the total daily intake, or a concentrate, metabolite, constituent, extract, or combinations of these ingredients that provides medical and/or health benefits. The medical and/or health benefits can include reducing the risk of a condition by decreasing joint inflammation.

The DHA can be formulated in a dosage form. These dosage forms can include, but are not limited to, tablets, capsules, cachets, pellets, pills, gelatin capsules, powders, and granules; and parenteral dosage forms which include, but are not limited to, solutions, suspensions, emulsions, coated particles, and dry powder comprising an effective amount of the DHA as taught in this invention. In some embodiments, the dosage form can be inserted or mixed into a food substance. Various substances are known in the art to coat particles, including cellulose derivatives, e.g., microcrystalline cellulose, methyl cellulose, carboxymethyl cellulose; polyalkylene glycol derivatives, e.g., polyethylene glycol; talc, starch, methacrylates, etc. In some embodiments, the dosage form is a capsule, wherein the capsule is filled with a solution, suspension, or emulsion comprising the DHA. It is also known in the art that the active ingredients can be contained in such formulations with pharmaceutically acceptable excipients such as diluents, fillers, disintegrants, binders, lubricants, surfactants, hydrophobic vehicles, water soluble vehicles, emulsifiers, buffers, humectants, moisturizers, solubilizers, preservatives, flavorants, taste-masking agents, sweeteners, and the like. Suitable excipients can include, e.g., vegetable oils (e.g., corn, soy, safflower, sunflower, or canola oil). In some embodiments, the preservative can be an antioxidant, e.g., sodium sulfite, potassium sulfite, metabisulfite, bisulfites, thiosulfates, thioglycerol, thiosorbitol, cysteine hydrochloride, α-tocopherol, and combinations thereof. The means and methods for administration are known in the art and an artisan can refer to various pharmacologic references for guidance. For example, “Modern Pharmaceutics,” Banker & Rhodes, Informa Healthcare, 4th ed. (2002); “Goodman & Gilman's The Pharmaceutical Basis of Therapeutics,” McGraw-Hill, New York, 10th ed. (2001); and Remington's Pharmaceutical Sciences, 20th Ed., 2001 can be consulted.

The DHA of the present invention is orally active and this route of administration can be used in the invention. Accordingly, administration forms can include, but are not limited to, tablets, dragees, capsules, caplets, gelatin capsules, and pills, which contain the DHA and one or more suitable pharmaceutically acceptable carriers.

For oral administration, the DHA can be administered as an oil or it can be formulated readily by combining it with a pharmaceutically acceptable carrier or with pharmaceutically acceptable carriers. Pharmaceutical acceptable carriers are well known in the art. Such carriers enable the compounds of the invention to be formulated as tablets, gelatin capsules, pills, dragees, capsules, liquids, gels, syrups, slurries, suspensions and the like, for oral ingestion by a subject to be treated. In some embodiments, the dosage form is a tablet, gelatin capsule, pill or caplet. Pharmaceutical preparations for oral use can be obtained by adding a solid excipient, optionally grinding the resulting mixture, and processing the mixture of granules, after adding suitable auxiliaries, if desired, to obtain tablets or dragee cores. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, vegetable oil (e.g., soybean oil), and polyvinylpyrrolidone (PVP). If desired, disintegrating agents can be added, such as, but not limited to, the cross-linked polyvinyl pyrrolidone, agar, or alginic acid or a salt thereof such as sodium alginate. Pharmaceutical preparations which can be used orally include, but are not limited to, push-fit capsules made of gelatin, as well as soft, sealed capsules made of gelatin and a plasticizer, such as glycerol or sorbitol. Capsule shells can be composed of non-animal derived ingredients, i.e., vegetarian ingredients, such as carrageenan, alginate, modified forms of starch, cellulose and/or other polysaccharides. All formulations for oral administration should be in dosages suitable for such administration.

It should be understood that in addition to the ingredients particularly mentioned above, the formulations of this invention can include other suitable agents such as flavoring agents, preservatives, and antioxidants. In particular, it is desirable to mix the microbial oils with an antioxidant to prevent oxidation of the DHA. Such antioxidants are pharmaceutically acceptable and can include vitamin E, carotene, BHT or other antioxidants known to those of skill in the art.

By way of example and not limitation, administration can be by parenteral, subcutaneous, intravenous (bolus or infusion), intramuscular, or intraperitoneal routes. Dosage forms for these modes of administration can include conventional forms, either as liquid solutions or suspensions, solid forms suitable for solution or suspension in liquid prior to injection, or as emulsions.

In some embodiments, the DHA can be provided in a dietary supplement, medical food or animal feed. “Dietary supplement” refers to a compound or composition used to supplement the diet of an animal or human. In some embodiments, the dietary supplement can further comprise various “dietary ingredients” intended to supplement the diet. “Dietary ingredients” can further include: vitamins, minerals, herbs or other botanicals, amino acids, and substances such as enzymes, organ tissues, glandulars, and metabolites. Dietary ingredients can also include extracts or concentrates. In some embodiments, the dosage form of DHA is administered in a dietary supplement.

The present invention is also directed to an oral dosage form consisting essentially of about 430 mg to about 12.4 g of docosahexaenoic acid (DHA) wherein the dosage form comprises less than about 1% eicosapentaenoic acid (EPA) and less than about 2% docosapentaenoic acid 22:5n-6 (DPAn6). In some embodiments, the oral dosage form is a unit dosage form, in particular, a gelatin capsule. Optionally the gelatin capsule also comprises a colorant, flavoring, and/or antioxidant.

The present invention includes gelatin capsules that are hard or soft gelatin capsules. In some embodiments, the encapsulating material comprises a gelatin, a plasticizer, and water. In certain embodiments, the encapsulating material is vegetarian, i.e., made from non-animal derived material, including plants, seaweed (for example, carrageenan), food starch, modified corn starch, potato starch, and tapioca. In other embodiments, the encapsulating material is derived from animals, including porcine, bovine, and fish-based materials, such as gelatins. Plasticizers of the invention include glycerin, glycerol, polyols, and mixtures thereof. In some embodiments, the plasticizer is a high boiling point polyol, such as glycerol or sorbitol.

In some embodiments, the gelatin capsule is a soft-gelatin capsule made from gelatin, glycerol, and water, and filled with DHA and an antioxidant. In certain embodiments, the gelatin capsule is animal or vegetable derived. In some embodiments, the gelatin capsule comprises a 0.5 gram dosage form, wherein the fill weight of the weight of the dosage form is from about 450 mg to about 550 mg, and wherein the gelatin capsule comprises from about 430 mg to about 480 mg DHA. In some embodiments, the gelatin capsule comprises about 450 mg DHA per 500 mg of the dosage form. In some embodiments, the gelatin capsule comprises about 450 mg DHA per 500 mg of the dosage form. In some embodiments, the gelatin capsule comprises a 1 gram dosage form, wherein the fill weight of the dosage form is from about 950 mg to about 1050 mg, and wherein the gelatin capsule comprises from about 860 mg to about 950 mg DHA per 1000 mg of the dosage form. In some embodiments, the gelatin capsule comprises about 900 mg DHA per 1,000 g of the dosage form.

In certain embodiments, the gelatin capsule is vegetarian. In some embodiments, the capsule preparation comprises no animal products, and comprises glycerol (and/or other polyols), seaweed extract (carrageenan) and water. In some embodiments, the water is purified. In some embodiments, color, flavor and/or sweeteners are added. During encapsulation, in some embodiments, fractionated coconut oil is used as a lubricant.

In some embodiments, the gelatin capsule comprises a capsule preparation, an active, and optionally a colorant and/or antioxidant. In another embodiment i) the capsule preparation comprises gelatin (bovine acid hide), glycerin, and purified water, ii) the active comprises DHA-EE, iii) the optional colorant is selected from titanium dioxide, FD&C Yellow #5, FD&C Red 40, and mixtures thereof; and iv) the antioxidant is ascorbyl palmitate. In some embodiments, the raw materials are USP raw materials.

In some embodiments, the gelatin capsules are soft gelatin capsules of about 1 g, having the specifications within the limits set forth in Table 7:

TABLE 7
Specifications for 1 gram DHA Ethyl Ester Gelatin Capsules

| Test | Specification |
|---|---|
| DHA EE Content, per capsule | 855-945 mg |
| Average Fill Weight | 950-1050 mg |
| Disintegration | Complies USP |
| Acid Value | Max 2 mg KOH/g |
| Peroxide Value (PV) | Max 10 meq/kg |
| Anisidine Value (AV) | Max 20 |
| Microbial Limits Tests | Complies with <61> USP |

Set forth in Table 8 is a list of components that are, in some embodiments, used in the manufacture of a DHA-EE soft gelatin capsule, and at least one corresponding function for each component.

TABLE 8
List of Components in 1 gram DHA
Ethyl Ester Soft Gelatin Capsules

| Component | Function |
|---|---|
| 900 mg DHA EE | Active |
| Gelatin, Bovine Acid Hide | Capsule Preparation |
| Glycerin | Capsule Preparation |
| Purified Water | Capsule Preparation |
| Titanium Dioxide | Colorant |
| FD&C Yellow #5 | Colorant |
| FD&C Red #40 | Colorant |

In some embodiments, the gelatin capsule is a soft-gelatin capsule made from gelatin, glycerol, and water, and filled with DHA and an antioxidant. In certain embodiments, the gelatin capsule is animal or vegetable derived. In some embodiments, the gelatin capsule comprises a 0.5 gram dosage form, wherein the fill weight of the weight of the dosage form is from about 450 mg to about 550 mg, and wherein the gelatin capsule comprises from about 430 mg to about 480 mg DHA. In some embodiments, the gelatin capsule comprises about 450 mg DHA per 500 mg of the dosage form. In some embodiments, the gelatin capsule comprises about 450 mg DHA per 500 mg of the dosage form. In some embodiments, the gelatin capsule comprises a 1 gram dosage form, wherein the fill weight of the dosage form is from about 950 mg to about 1050 mg, and wherein the gelatin capsule comprises from about 860 mg to about 950 mg DHA per 1000 mg of the dosage form. In some embodiments, the gelatin capsule comprises about 900 mg DHA per 1,000 g of the dosage form.

In certain embodiments, the gelatin capsule is vegetarian. In some embodiments, the capsule preparation comprises no animal products, and comprises glycerol (and/or other polyols), seaweed extract (carrageenan) and water. In some embodiments, the water is purified. In some embodiments, color, flavor and/or sweeteners are added. During encapsulation, in some embodiments, fractionated coconut oil is used as a lubricant.

In some embodiments, the gelatin capsule comprises a capsule preparation, an active, and optionally a colorant and/or antioxidant. In another embodiment i) the capsule preparation comprises gelatin (bovine acid hide), glycerin, and purified water, ii) the active comprises DHA-EE, iii) the optional colorant is selected from titanium dioxide, FD&C Yellow #5, FD&C Red 40, and mixtures thereof; and iv) the antioxidant is ascorbyl palmitate. In some embodiments, the raw materials are USP raw materials. In some embodiments, the gelatin capsules are soft gelatin capsules of about 1 g, having the specifications within the limits set forth in Table 9:

TABLE 9
Specifications for 1 gram DHA Ethyl Ester Gelatin Capsules

| TEST | SPECIFICATION |
|---|---|
| DHA EE CONTENT, PER CAPSULE | 855-945 MG |
| AVERAGE FILL WEIGHT | 950-1050 MG |
| DISINTEGRATION | COMPLIES USP |
| ACID VALUE | MAX 2 MG KOH/G |
| PEROXIDE VALUE (PV) | MAX 10 MEQ/KG |
| ANISIDINE VALUE (AV) | MAX 20 |
| MICROBIAL LIMITS TESTS | COMPLIES WITH <61> USP |

Set forth in Table 10 is a list of components that are, in some embodiments, used in the manufacture of a DHA-EE soft gelatin capsule, and at least one corresponding function for each component.

TABLE 10
List of Components in 1 gram DHA
Ethyl Ester Soft Gelatin Capsules

| COMPONENT | FUNCTION |
|---|---|
| 900 MG DHA EE | ACTIVE |
| GELATIN, BOVINE ACID HIDE | CAPSULE PREPARATION |
| GLYCERIN | CAPSULE PREPARATION |
| PURIFIED WATER | CAPSULE PREPARATION |
| TITANIUM DIOXIDE | COLORANT |
| FD&C YELLOW #5 | COLORANT COLORANT |
| FD&C RED #40 | |

As used herein, “arthritis” refers to inflammatory diseases of the joints, including, but not limited to osteoarthritis, gouty arthritis, ankylosing spondylitis, psoriatic arthritis, reactive arthritis, rheumatoid arthritis, juvenile onset rheumatoid arthritis, infectious arthritis, inflammatory arthritis, septic arthritis, degenerative arthritis, arthritis mutilans, and lyme arthritis

The present invention is directed to methods of treating arthritis. “Inflammation” can refer to a fundamental pathological process consisting of a dynamic complex of cytological and chemical reactions that occur in the affected blood vessels and adjacent tissues in response to an injury or abnormal stimulation caused by a physical, chemical or biological agent. “Inflammation” can also refer to pain, swelling, or stiffness resulting from infection, trauma, or degenerative changes.

The present invention is also directed to methods for improving joint performance, function, and health.

In some embodiments of the present invention, DHA treatment results in a greater decrease in arthritis severity scores, a greater decrease in arthritis severity/area under curve, a decrease in histopathology parameters associated with arthritis (inflammation, pannus, cartilage damage, and bone damage), a decrease in serum arachidonic acid levels, or a decrease in anti-collagen antibodies, relative to administration of (a) a composition comprising DHA and EPA and/or (b) a composition comprising DHA and DPAn6; and/or (c) a composition comprising DHA, EPA, and DPAn6.

The method of the present invention can be administered to individuals who have arthritis or individuals who are at risk for developing arthritis. Thus, in some embodiments the invention is directed to a method of treating a subject having normal joints, borderline arthritic joints, or very arthritic joints, the method comprising administering the DHA as described herein. In some embodiments, the method of the present invention can be used to treat chronic arthritis for the remainder of the life of the subject.

The term “administering” or “administration” of the composition refers to the application of the composition, e.g., oral or parenteral (e.g., transmucosal, intravenous, intramuscular, subcutaneous, rectal, intravaginal, or via inhalation) to the subject. Administering would also include the act of prescribing a composition described herein to a subject by a medical professional for treatment of arthritis. Administering can also include the act of labeling a composition, i.e., instructing a subject to administer a composition, in a manner as provided herein for treatment of arthritis. In some embodiments, the term “administering” or “administration” of the composition refers to the application of the composition, e.g., oral or parenteral (e.g., transmucosal, intravenous, intramuscular, subcutaneous, rectal, intravaginal, or via inhalation) to the subject. Administering would also include the act of prescribing a composition described herein to a subject by a medical professional for treatment of arthritis. Administering can also include the act of labeling a composition, i.e., instructing a subject to administer a composition, in a manner as provided herein for treatment of arthritis

The terms “treat” and “treatment” refer to both therapeutic treatment and prophylactic or preventative measures, wherein the object is to prevent or slow down (lessen) an undesired physiological condition, disorder or disease, or obtain beneficial or desired clinical results. For purposes of this invention, beneficial or desired clinical results include, but are not limited to, alleviation of symptoms associated with arthritis; prevention of arthritis; delay in the onset of arthritis; reduced incidence of arthritis in a population; diminishment of the extent of the condition associated with arthritis; stabilization (i.e., not worsening) of the state of the condition, disorder or disease associated with arthritis; delay in onset or slowing of the condition, disorder or disease progression associated with arthritis; amelioration of the condition, disorder or disease state, remission (whether partial or total) of the condition, disorder or disease associated with arthritis, whether detectable or undetectable; or enhancement or improvement of the condition, disorder or disease associated with arthritis. The term “preventing” means to stop or hinder a disease, disorder, or symptom of a disease or condition. Treatment includes eliciting a clinically significant response, without excessive levels of side effects. Treatment also includes prolonging survival as compared to expected survival if not receiving treatment.

The term “subject” refers to mammals such as humans or primates, such as apes, monkeys, orangutans, baboons, gibbons, and chimpanzees. The term “subject” can also refer to companion animals, e.g., dogs and cats; zoo animals; equids, e.g., horses; food animals, e.g., cows, pigs, and sheep; and disease model animals, e.g., rabbits, mice, and rats. The subject can be a human or non-human. The subject can be of any age. By way of example and not limitation, in some embodiments, the subject is a human infant, i.e., post natal to about 1 year old; a human child, i.e., a human between about 1 year old and 12 years old; a pubertal human, i.e., a human between about 12 years old and 18 years old; or an adult human, i.e., a human older than about 18 years old. In some embodiments, the subject is an adult, either male or female. In some embodiments, the subject is a patient.

In some embodiments, the subject is a “subject in need thereof.” A subject in need thereof refers to an individual for whom it is desirable to treat, i.e., to reduce inflammation of the joints, prevent inflammation of the joints, or retard the onset of inflammation of the joints.

Administration of the DHA dosage forms of the present invention can be achieved using various regimens. For example, in some embodiments, administration of the DHA dosage forms is daily on consecutive days, or alternatively, the dosage form is administered every other day (bi-daily). Administration can occur on one or more days. In some embodiments, administration of the DHA dosage form continues even after the symptoms of arthritis have been alleviated. In some embodiments, the administration of the DHA is administered as a prophylactic measure, before the onset of symptoms associated with arthritis.

The dosage forms of the present invention can be administered for a long duration of time or a short duration of time. For example, in some embodiments, the subject has a chronic condition, and is administered the DHA of the present invention for the remainder of the subject's lifetime, or for more than 6 months, more than one year, more than 2 years, more than 5 years, more than 10 years, or more than 20 years. In some embodiments, DHA of the present invention is administered daily for a shorter duration, e.g., 1 week to 12 weeks.

In some embodiments, the DHA is administered continuously. The term “continuous” or “consecutive,” as used herein in reference to “administration,” means that the frequency of administration is at least once daily. Note, however, that the frequency of administration can be greater than once daily and still be “continuous” or “consecutive,” e.g., twice or even three or four times daily, as long as the dosage levels as specified herein are achieved.

Administration of DHA dosage forms can be combined with other regimens (i.e., non-DHA regimens) used to treat arthritis. By way of example and not limitation, the method of the present invention can be combined with diet regimens (e.g., low carbohydrate diets, high protein diets, high fiber diets, etc.), exercise regimens, or weight loss regimens, to treat arthritis. The methods of the present invention can also be used in combination with other pharmaceutical products to treat arthritis in a subject. Non-DHA regimens can also include other pharmaceutical products including, e.g., nonsteroidal anti-inflammatory drugs (NSAIDs), cox-2 inhibitors, steroids, narcotic pain relievers, disease-modifying antirheumatic drugs (DMARDs), TNF blockers, biological response modifiers, chemotherapy medications, and gout medicines. Specific treatments that can be used in a combination DHA regimen include, e.g., aspirin, hydroxychloroquine, gold, penicillamine, azathioprine, sulfasalazine, methotrexate, hyaluronan, anakinra, adalimumab, abatacept, rituximab, pregabalin, duloxetine HCl, milnacipran HCl, febuxostat, golimumab, certolizumab pegol, infliximab, etanercept, chrondroitin, and glucosamine.

In some embodiments, the DHA of the present invention are administered before the non-DHA regimens. Alternatively, in some embodiments, the non-DHA regimens are administered first, and then the DHA dosage forms of the present invention are administered. In some embodiments, the DHA regimen and the non-DHA regimen are administered at the same time.

The present invention is directed to kits or packages comprising one or more dosage forms to be administered according to the methods of the present invention. A kit or package can contain one dosage form, or more than one dosage form (i.e., multiple dosage forms). If multiple dosage forms are present in the kit or package, the multiple dosage forms can be optionally arranged for sequential administration. The kits can contain dosage forms of a sufficient number to provide convenient administration to a subject who has a chronic condition and requires long-term administration of the DHA of the present invention. For example, in some embodiments, the kit provides dosage forms of a sufficient number for 1, 2, 3 or 4 months of daily administration of the DHA. In some embodiments of the present invention, the kit comprises dosage forms for shorter periods of administration, e.g., the kit can contain about 7, 14, 21, 28 or more dosage forms for oral administration, each dosage form comprising about 450 mg to about 12.05 g DHA and intended for ingestion on successive days.

The kits of the present invention can optionally contain instructions associated with the dosage forms of the kits. Such instructions can be in a form prescribed by a governmental agency regulating the manufacture, use or sale of pharmaceutical products, which notice reflects approval by the agency of the manufacture, use or sale for human administration to treat a condition or disorder. The instructions can be in any form which conveys information on the use of the dosage forms in the kit according to the methods of the invention. By way of example and not limitation, the instructions can be in the form of printed matter, or in the form of a pre-recorded media device.

In the course of examination of a patient, a medical professional can determine that administration of one of the methods of the present invention is appropriate for the patient, or the physician can determine that the patient's condition (e.g., the patient may be suffering from arthritis) can be improved by the administration of one of the methods of the present invention. Prior to prescribing any DHA regimen, the physician can counsel the patient, for example, on the various risks and benefits associated with the regimen. The patient can be provided full disclosure of all the known and suspected risks associated with the regimen. Such counseling can be provided verbally, as well as in written form. In some embodiments, the physician can provide the patient with literature materials on the regimen, such as product information, educational materials, and the like.

The present invention is also directed to methods of educating consumers about the methods of lowering triglyceride levels of the present invention, the method comprising distributing the DHA dosage forms with consumer information at a point of sale. In some embodiments, the distribution will occur at a point of sale having a pharmacist or healthcare provider.

The term “consumer information” can include, but is not limited to, an English language text, non-English language text, visual image, chart, telephone recording, website, and access to a live customer service representative. In some embodiments, consumer information will provide directions for use of the DHA unit dosages according to the methods of the present invention, appropriate age, use, indication, contraindications, appropriate dosing, warnings, telephone number, and website address. In some embodiments, the method further comprises providing professional information to relevant persons in a position to answer consumer questions regarding use of the disclosed regimens according to the methods of the present invention. The term “professional information” includes, but is not limited to, information concerning the regimen when administered according to the methods of the present invention that is designed to enable a medical professional to answer customer questions.

A “medical professional,” includes, by way of example and not limitation, a physician, physician assistant, nurse practitioner, pharmacist and customer service representative. All of the various aspects, embodiments and options described herein can be combined in any and all variations.

### EXAMPLES

### Example 1

### The Effects DHA, DPAn6, DHA/DPAn6 and DHA/EPA Ethyl Esters on Murine Collagen-Induced Arthritis (CIA-EE)

The anti-arthritic activity of DHA, DPAn6, DHA/DPAn6 (DHA S-oil ratio) and DHA/EPA (fish oil ratio) ethyl esters (EE) in a chronic mouse model of inflammation, Collagen-Induced Arthritis (CIA), was studied.

Male DBA/1O1aHsd mice (3.5-4 weeks of age) were randomized by weight and enrolled in the study after a three day quarantine in which animals were fed the AlN-93G diet with a modified base fat: 1 part flax seed oil (flax), 9 parts hydrogenated coconut oil (HCOC) and 21 parts high-oleic safflower oil (HO-SAFF). 15 animals were enrolled per study group, with the exception of the No Disease control that had 4 animals. Study groups consisted of:

### Controls:

No Disease (negative control)

Dexamethasone treatment (positive control, 0.1 mg/kg)

Oleic acid (OA)-EE (placebo control) (2500 mg/kg or 5000 mg/kg)

Treatment Groups of Ethyl Esters (2500 mg/kg or 5000 mg/kg PUFA dose):

DHA-EE

DPAn6-EE

DHA/DPAn6-EE (2.5:1 ratio)

DHA/EPA-EE (1:1.5 ratio)

The appropriate ethyl esters were blended into the base diet to provide approximately 2500 and 5000 mg/kg polyunsaturated fatty acid (PUFA) per day. The human equivalent doses (HED) were approximately 12 g/day and 22 g/day, respectively. The 2500 mg/kg EE diets contained the following fat blend: 1 part flax, 12 parts HCOC and 23 parts HO-SAFF. The 5000 mg/kg EE diets contained a fat blend consisting of: 1 part flax, 5 parts HCOC and 20 parts HO-SAFF. All study diets provided a total of 7% fat. Animals were fed study diets for approximately 30 days prior to the first collagen injection at day 0 and remained on the diets until study termination (day 45). Animals in the No Disease control and Dexamethasone groups were fed the diet containing OA-EE. Following a second collagen injection on day 21, arthritis severity was scored daily. Blood samples were collected at day 21 and day 45. Joints, 6 per an animal, were collected for histology and pathology on day 45.

### Results

Statistical Analyses:

Data was analyzed and compared by one-way ANOVA using the Bonferroni post-test to compare treatment groups to respective placebo control and to make comparisons between treatment groups (GraphPad, Prism, Version 4.0). Effects were considered to be statistically significant at p<0.05.

Food Intake:

There were no significant differences in food intake between study groups (data not shown). The animal received PUFA doses averaging approximately 2400 and 4700 mg/kg/day.

Body Weight:

There were no significant differences in body weight gain or loss between groups (data not shown).

Clinical Arthritis:

Arthritis severity was assessed from day 18 to 45; a score of 0 represented an animal devoid of clinical arthritis symptoms and a score of 5 represented advanced clinical arthritis. Scores from 0-5 (most severe) were assigned to each digit on the fore and hind limbs based on erythema, swelling and mobility and the mean arthritis severity per animal was determined.

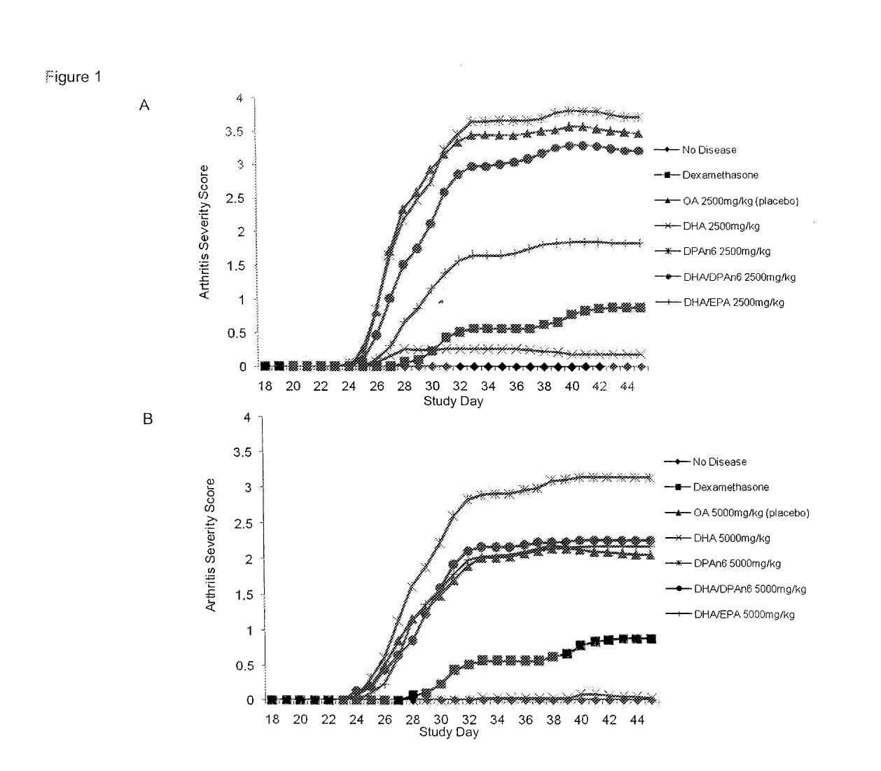

DHA-EE treatment reduced arthritis severity at both the 2500 mg/kg and 5000 mg/kg doses (FIG. 1A/B). The arthritis severity score was also calculated for study days 18-45 as area under the curve (AUC) (FIG. 2). DHA-EE treatment significantly reduced arthritis severity at both the 2500 mg/kg and 5000 mg/kg doses (p<0.001 and p<0.01, respectively), as compared to the appropriate OA-EE (placebo) control. DHA-EE was as effective as dexamethasone, a drug, at reducing arthritis severity to levels comparable to normal, non-diseased animals. DHA/EPA-EE at 2500 mg/kg, although effective at reducing arthritis (p<0.01), as compared to 2500 mg/kg OA-EE (placebo) control, was not as potent as DHA-EE alone. DHA-EE was more potent than DHA/EPA-EE at 5000 mg/kg (p<0.001); 5000 mg/kg DHA/EPA-EE did not significantly reduce arthritis severity. DPAn6-EE and DHA/DPAn6-EE did not reduce arthritis at either dose. Means (n=15)±Standard Deviation (SD). **p<0.01 ***p<0.001.

Incidence:

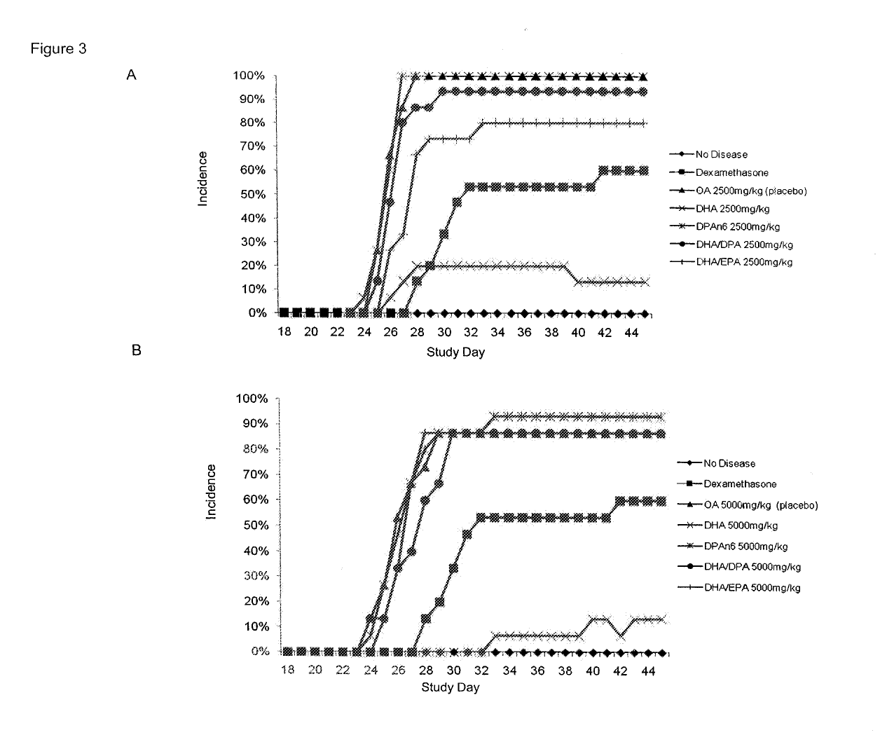

The incidence of disease was reduced in animals treated with DHA-EE (FIG. 3A/B). A clinical arthritis score greater than 0 was considered diseased. The incidence of disease at day 45 was 13% in animals treated with 2500 mg/kg DHA-EE, as compared to 100% disease incidence in OA-EE (placebo) control animals. Treatment with DHA/EPA-EE resulted in 80% incidence. DPAn-6-EE or DHA/DPAn6-EE treatment did not reduce disease incidence (100% and 93% incidence, respectively). Dexamethasone treatment reduced disease incidence to 60%. Similar results were seen at the 5000 mg/kg dose (FIG. 3B).

Histopathology:

Placebo-treated diseased animals had histopathology changes, consistent with those seen in type II collagen induced arthritis, in most joints, with scores ranging from normal to severe. Microscopic alteration included infiltration of synovium and periarticular tissue with neutrophils and mononuclear inflammatory cells (inflammation), marginal zone pannus and bone resorption and cartilage damage (proteoglycan loss, chondrocyte death and collagen matrix destruction). Four-paw, knee and joint parameters were assigned a quantitative score 0=normal, 1=minimal, 2=mild, 3=moderate, 4=marked, 5=severe and a total score was determined per animal.

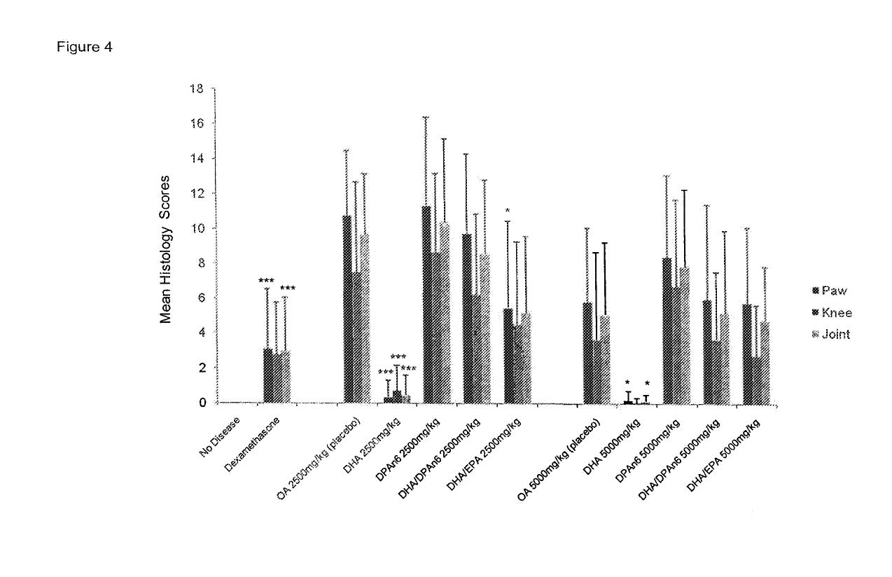

All paw histopathology parameters (inflammation, pannus, cartilage damage, bone damage) were significantly reduced by treatment with DHA-EE 2500 mg/kg (p<0.001), DHA/EPA-EE 2500 mg/kg (p<0.05) or Dexamethasone (p<0.001), as compared to OA-EE 2500 mg/kg (placebo) control (FIG. 4). Treatment with 5000 mg/kg DHA-EE also reduced paw histopathology scores (p<0.05), whereas DHA/EPA-EE had no significant effect, as compared to 5000 mg/kg OA-EE (placebo) control. DPAn6-EE and DHA/DPAn-6-EE did not significantly affect paw histopathology at the doses examined. Means (n=15)±SD. *p<0.05**p<0.01 ***p<0.001.

Knee histopathology parameters were significantly reduced by treatment with 2500 mg/kg DHA-EE (p<0.001), as compared to 2500 mg/kg OA-EE (placebo) control (p<0.001) (FIG. 4). Treatment with DHA/EPA-EE, DPAn6-EE DHA/DPAn6-EE, or Dexamethasone did not significantly improve knee histopathology at either dose examined. Means (n=15)±SD. *p<0.05**p<0.01***p<0.001.

All six-joint mean histopathology parameters were significantly reduced by treatment with 2500 mg/kg or 5000 mg/kg DHA-EE (p<0.001 and p<0.05, respectively) or Dexamethasone (p<0.001) as compared to 2500 mg/kg OA-EE (placebo) control (FIG. 4). Means (n=15)±SD. *p<0.05**p<0.01***p<0.001.

Serum Phospholipid FAME:

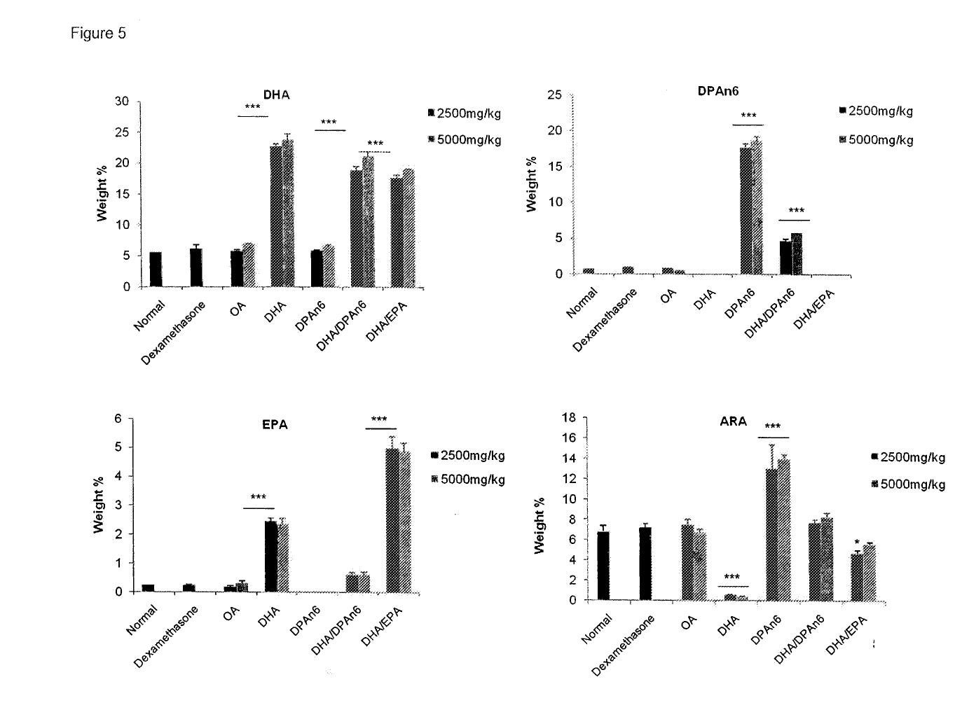

Serum phospholipid fatty acid levels were determined by FAME analysis (FIG. 5). Treatment with DHA-EE elevated serum DHA levels 3-4 fold, as compared the OA-EE (placebo) control, whereas DPAn6-EE elevated serum DPAn6 20-35 fold. Serum EPA levels were elevated when dosed with the DHA/EPA-EE blend 8-15 fold and 4-7 fold when dosed with DHA-EE, as compared to the OA-EE (placebo) control. Reductions in serum ARA levels appeared to parallel with disease severity; ARA levels were decreased approximately 90% in animals treated with 2500 or 5000 mg/kg DHA-EE (p<0.001), as compared to the appropriate OA-EE (placebo) control (FIG. 5). Treatment with 2500 mg/kg DHA/EPA-EE reduced ARA levels to a lesser degree (36%, p<0.05), as compared to the 2500 mg/kg OA-EE (placebo) control; however, ARA levels were not significantly affected at 5000 mg/kg DHA/EPA-EE. Data shown represents 3 pooled animals per group. Means±SD. *p<0.05***p<0.001.

Anti-Collagen Antibodies:

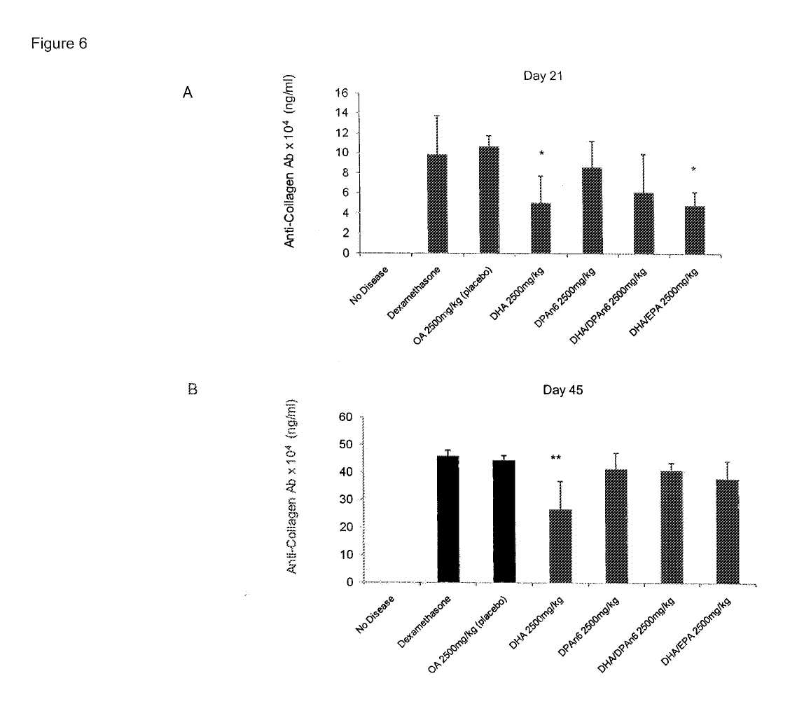

DHA-EE significantly reduced the level of plasma anti-collagen antibodies (FIG. 6A/B). Anti-collagen antibody production was measured prior to the antigen challenge (day 21) and at study termination (day 45). DHA-EE treatment reduced the level of anti-collagen antibodies at both the 2500 mg/kg and 5000 mg/kg doses (p<0.05 and p<0.01, respectively), as compared to the appropriate OA-EE (placebo) control. DHA/EPA-EE (2500 mg/kg) also reduced anti-collagen antibody production at day 21 (p<0.05), as compared to the 2500 mg/kg OA-EE (placebo) control; however, this reduction was transient and no significant effect was seen at day 45. Means (n=5)±SD. *p<0.05**p<0.01.

To summarize, this study showed that DHA, when provided as an ethyl ester (2500 and 5000 mg/kg), significantly reduced arthritis severity in the mouse 45 day developing type II collagen arthritis model. DHA-EE treatment significantly reduced terminal serum ARA levels, as well as levels of anti-collagen antibodies, which may play a role in disease pathogenesis. DHA-EE was as efficacious as the drug Dexamethasone at reducing arthritis severity. Further, DHA-EE was more potent than the fish oil mimic DHA/EPA-EE combination at reducing arthritis in this model.

### Example 2

### Comparison of the Anti-Inflammatory Activity of Ethyl Esters (EE) and Triglycerides (TG) of DHA, DHA/DPAn6, and DHA/EPA in a Prophylactic Mouse Model of Collagen-Induced Arthritis (CIA EE Vs. TG)

The anti-arthritic activity of DHA, DHA/DPAn6 and DHA/EPA, administered as EE or triglyceride oils (TG), in a chronic mouse model of inflammation, Collagen-Induced Arthritis (CIA), was studied.

Methods used in this study were similar to those described in Example 1 (CIA-EE Study). Study groups consisted of:

### Controls:

No Disease (negative control)

Dexamethasone (positive control, 0.1 mg/kg)

Placebo EE: Oleic acid (OA) (2500 mg/kg)

Placebo TG: monounsaturate control (2500 mg/kg)

Treatment Groups (2500 mg/kg PUFA):

DHA-EE

DHA/DPAn6-EE (2.5:1 ratio)

DHA/EPA-EE (1:1.5 ratio)

DHASCO® (DHA-TG) oil

DHA-S (DHA/DPAn6-TG) oil

Fish (Nordic Naturals, DHA/EPA-TG, 1:1.5 ratio) oil

Treatment Group (1000 mg/kg PUFA):

DHA-EE

Male DBA/1O1aHsd mice were enrolled, quarantined, and feed study diets following the protocols described in Example 1 (CIA-EE Study). EE and TG oils were blended into the base diet to provide 2500 mg/kg PUFA per day (HED approximately 12 g/day). A single dose of PUFA per day (HED approximately 5 g/day). The 1000 mg/kg DHA-EE dose was equivalent to the amount of DHA administered in the DHA/EPA-EE blend. The fat blend the 2500 mg/kg EE diets consisted of approximately: 1 part flax, 12 parts HCOC and 23 parts HO-SAFF; 1000 mg/kg EE diet: 1 part flax, 12 parts HCOC and 32 parts HO-SAFF; 2500 mg/kg DHASCO® oil diet: 1 part flax, 8 parts HCOC, 17 parts HO-SAFF and 15 parts soybean oil; 2500 mg/kg DHA-S oil diet: 1 part flax, 5 parts HCOC, 16 parts HO-SAFF and 6 parts soybean oil. 2500 mg/kg DHA/EPA (Nordic Naturals fish oil) diet: 1 part flax, 21 parts HCOC, 18 parts HO-SAFF and 13 parts soybean oil. The monounsaturate control animals received a fat blend of 1 part flax, 7 parts HCOC and 25 parts HO-SAFF. All study diets provided a total of 7% fat. Animals in the No Disease control and Dexamethasone groups were fed the diet containing OA-EE. Mice received collagen injections on day 0 and day 21, as described previously. Blood samples were collected at day 21 and day 45. At day 45, joints were collected for histology and pathology.

### Results

Statistical Analyses:

Data was analyzed and compared by one-way ANOVA using the Bonferroni post-test to compare treatment groups to respective placebo control and to make comparisons between treatment groups (GraphPad, Prism, Version 4.0). Effects were considered to be statistically significant at p<0.05.

Food Intake:

There were no significant differences in food intake between study groups. The average doses received were approximately 2400 and 950 mg/kg/day PUFA (for the 2500 and 1000 mg/kg target doses, respectively).

Body Weight:

There were no significant differences in body weight gain or loss between groups.

Clinical Arthritis:

Arthritis severity was assessed from day 18 to 45; a score of 0 represented an animal devoid of clinical arthritis symptoms and a score of 5 represented advanced clinical arthritis. Scores from 0-5 (most severe) were assigned to each digit on the fore and hind limbs based on erythema, swelling and mobility the mean arthritis severity per animal was determined.

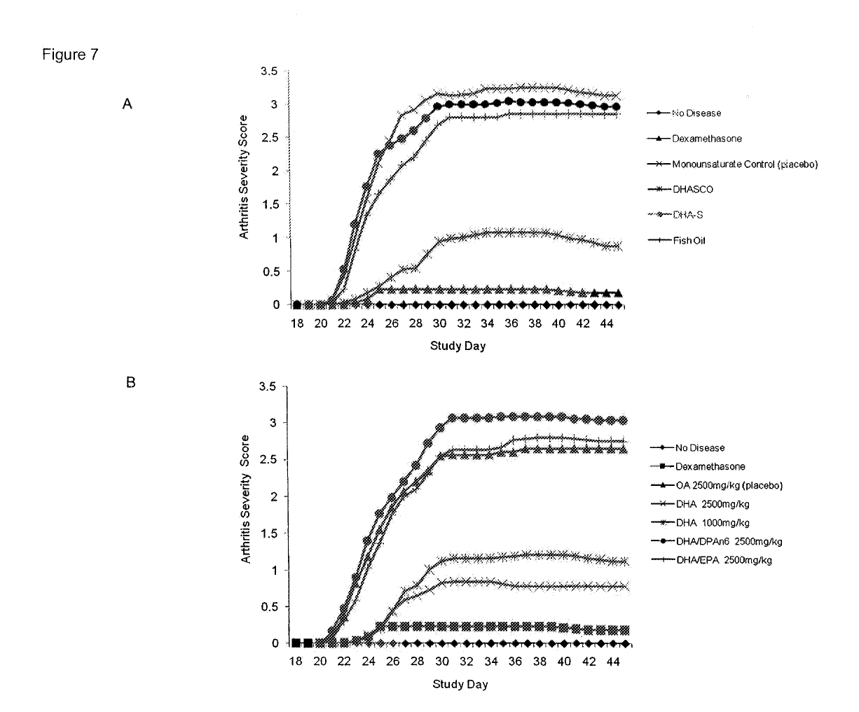

Treatment with DHASCO® or DHA-EE reduced arthritis severity (FIG. 7A/B). The arthritis severity score was also calculated for study days 18-45 as area under the curve (AUC) (FIG. 8A/B). DHASCO® treatment significantly reduced arthritis severity, as compared to the monounsaturated (placebo) control (p<0.001), and was as efficacious as Dexamethasone, a drug (FIG. 8A). Fish oil and DHA-S did not significantly reduce arthritis. When provided as EE, 1000 and 2500 mg/kg DHA significantly reduced arthritis (p<0.05 and p<0.01, respectively), as compared to the appropriate OA-EE (placebo) control (FIG. 8B). DHASCO® was more potent than fish oil at reducing arthritis severity (p<0.01); similarly DHA-EE (2500 mg/kg) was more potent than DHA-EPA-EE (p<0.01). DHA/EPA-EE and DHA/DPAn6-EE did not significantly reduce arthritis. Means (n=15)±SD. *p<0.05**p<0.01 ***p<0.001.

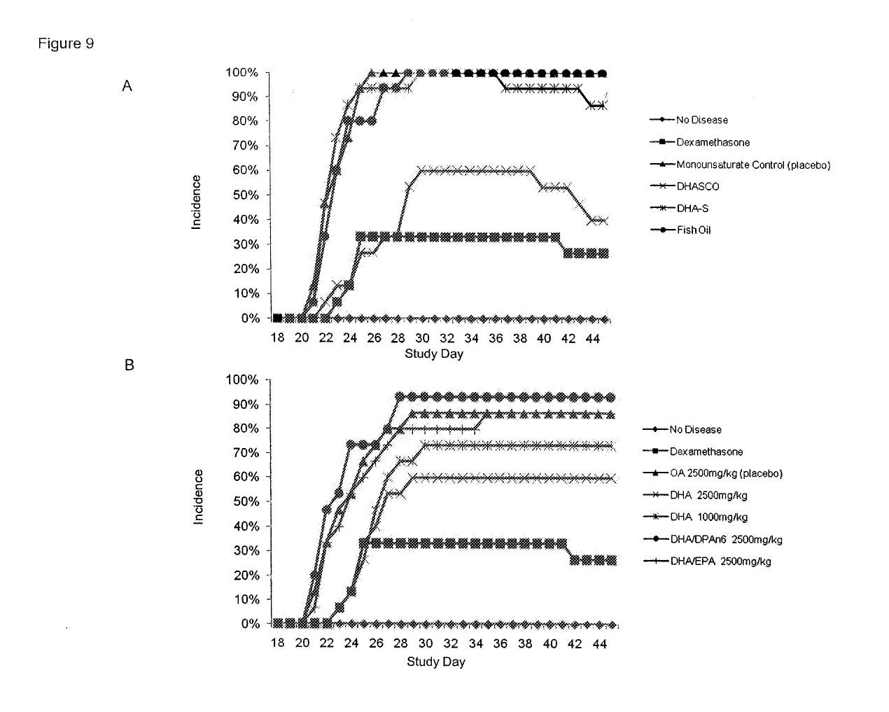

Incidence: The incidence of disease was reduced in animals treated with DHASCO® or DHA-EE at day 45. The incidence of disease at day 45 was 40% in DHASCO-treated animals, as compared to 100% in the monounsaturate (placebo) control group (FIG. 9A). Incidence was reduced to 87% with DHA-S treatment, whereas fish oil had no effect (100%). Similarly, 1000 mg/kg and 2500 mg/kg DHA-EE reduced disease incidence to 73% and 60%, respectively, as compared to the OA (placebo) control (87%) (FIG. 9B). DHA-S and fish oil reduced disease incidence (93% and 87%, respectively).

Histopathology:

Placebo-treated diseased animals had histopathology changes, consistent with those seen in type II collagen induced arthritis, in most joints, with scores ranging from normal to severe. Microscopic alteration included infiltration of synovium and periarticular tissue with neutrophils and mononuclear inflammatory cells (inflammation), marginal zone pannus and bone resorption and cartilage damage (proteoglycan loss, chondrocyte death and collagen matrix destruction). Four-paw, knee and joint parameters were assigned a quantitative score 0=normal, 1=minimal, 2=mild, 3=moderate, 4=marked, 5=severe and a total score was determined per animal.

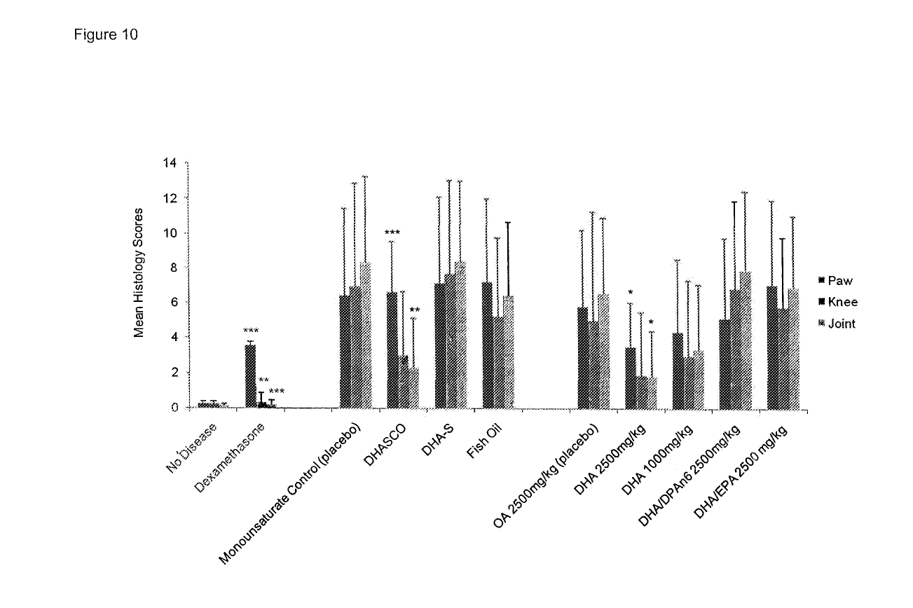

All paw histopathology parameters (inflammation, pannus, cartilage damage, bone damage) were reduced by treatment with DHASCO® (p<0.001), 2500 mg/kg DHA-EE (p<0.05), or Dexamethasone (p<0.001) (FIG. 10), as compared the appropriate monounsaturate or OA-EE (placebo) control. TG and EE treatments had no significant effect on knee histopathology parameters; parameters were reduced by treatment with Dexamethasone only (p<0.01), as compared to the monounsaturate (placebo) control (FIG. 10). Six-joint histopathology parameters were significantly reduced towards normal by treatment with DHASCO® (p<0.01), 2500 mg/kg DHA-EE (p<0.05), and Dexamethasone (p<0.001) (FIG. 10), as compared to the appropriate monounsaturate or OA-EE (placebo) control. Paw, knee, and six-joint histopathology was not significantly affected by DHA-S, fish oil, 1000 mg/kg DHA-EE, DHA/DPAn6-EE or DHA/EPA-EE treatments. Means (n=15)±SD. *p<0.05 **p<0.01***p<0.001.

Serum Phospholipid FAME Results:

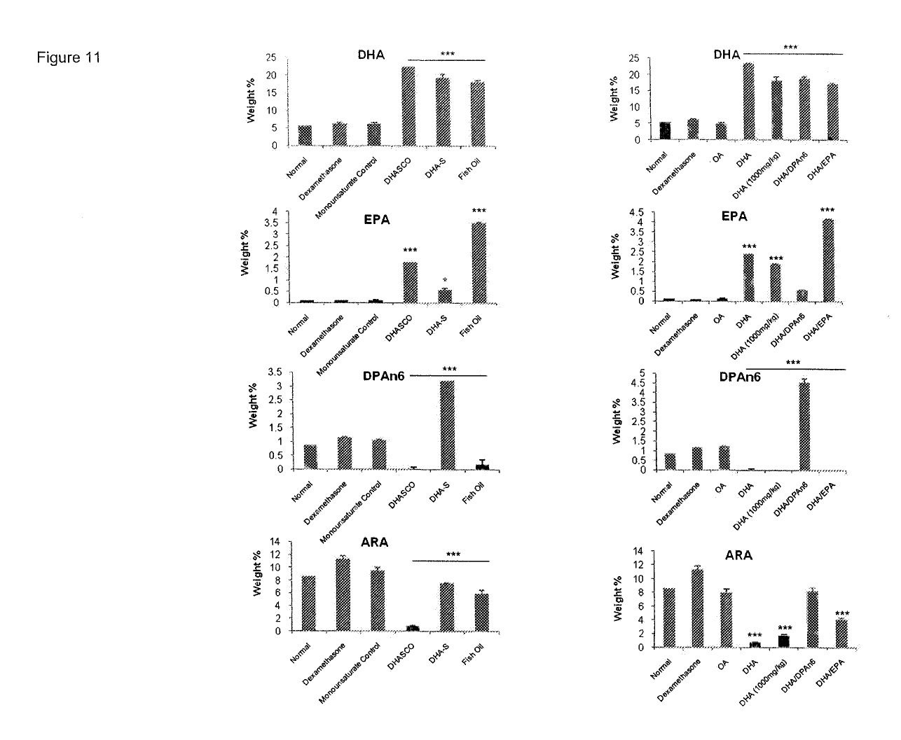

Serum phospholipid fatty acid levels were determined by FAME analysis (FIG. 11). Treatment with DHASCO® or DHA-S oil elevated serum DHA levels 3-4 fold (p<0.001), as compared to the monounsaturated (placebo) control. DHA-EE treatment elevated DHA levels 3-5 fold (p<0.001), as compared to the OA-EE (placebo) control. DPAn6-TG and EE elevated DPAn6 levels 3-4 fold (p<0.001), as compared to the monounsaturated or OA-EE (placebo) control. Serum EPA levels were elevated approximately 30 fold (p<0.001) when fish oil or DHA/EPA-EE was administered, as compared to the monounsaturated or OA-EE (placebo) control. Serum EPA levels were also elevated by treatment with DHASCO® (p<0.001), DHA-S (p<0.05), or with DHA-EE (p<0.001), as compared to the monounsaturated or OA-EE (placebo) control. ARA serum levels were found to parallel disease severity scores. Treatment with either DHASCO® or DHA-EE (2500 mg/kg) reduced ARA levels by approximately 90% (p<0.001), as compared to the monounsaturated or OA-EE (placebo) control. Treatment with 1000 mg/kg DHA-EE resulted in a 77% reduction (p<0.001), as compared to the OA-EE (placebo) control. Serum ARA levels were reduced to a lesser degree by DHA-S (24%, p<0.001), fish oil (37%, p<0.001) and DHA/EPA-EE (49%, p<0.001), as compared to the monounsaturated or OA-EE (placebo) control. Data shown represents 3 pooled animals per group. Means±SD. *p<0.05 ***p<0.001.

Anti-Collagen Antibodies:

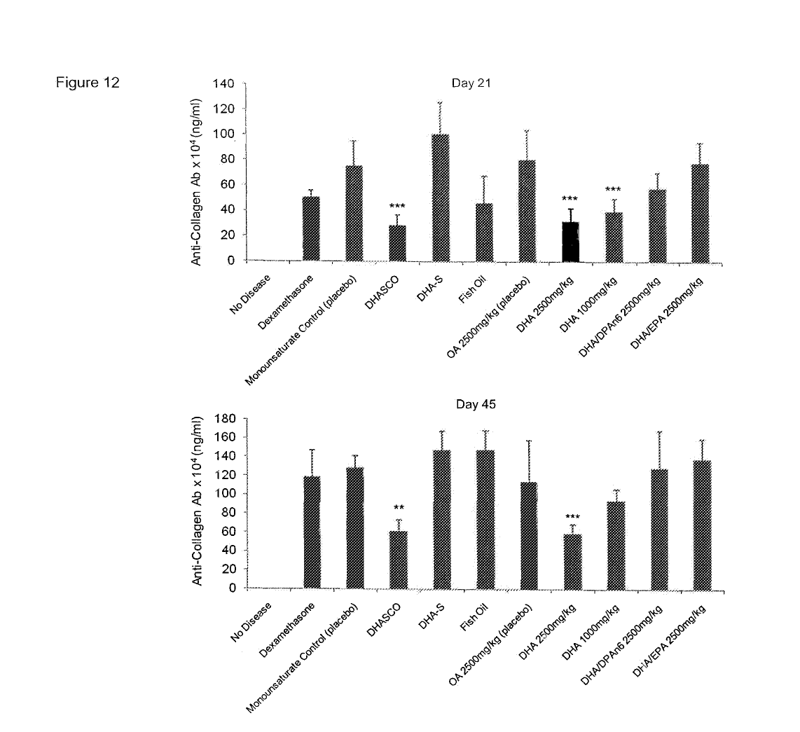

A reduction in serum anti-collagen antibodies also correlated with a reduction in disease severity. Treatment with DHASCO® or DHA-EE (1000 and 2500 mg/kg) significantly reduced serum anti-collagen antibodies at day 21 (p<0.001), as compared to the monounsaturated or OA-EE (placebo) control. (FIG. 12). At day 45, both DHASCO® and 2500 mg/kg DHA-EE significantly reduced serum anti-collagen antibody levels (p<0.01 and p<0.001, respectively), as compared to the monounsaturated or OA-EE (placebo) control. No significant reductions in serum anti-collagen antibodies were seen with DHA-S, fish oil, DHA/DPAn6-EE, or DHA/EPA-EE treatment.

To summarize, this study demonstrated that DHA, when provided as a TG or EE, significantly reduced arthritis severity in the mouse 45 day developing type II collagen arthritis model. Treatment with either DHA-TG or DHA-EE significantly reduced serum phospholipid ARA levels, as well as levels of anti-collagen antibodies that may play a role in disease pathogenesis. DHA, when administered as a TG or EE, was as efficacious as the drug Dexamethasone at reducing arthritis severity. Further, DHA-TG or DHA-EE was more potent than fish oil or the DHA/EPA-EE (fish oil mimic) at reducing arthritis in this model.

### CONCLUSION

All of the various embodiments or options described herein can be combined in any and all variations. While the invention has been particularly shown and described with reference to some embodiments thereof, it will be understood by those skilled in the art that they have been presented by way of example only, and not limitation, and various changes in form and details can be made therein without departing from the spirit and scope of the invention. Thus, the breadth and scope of the present invention should not be limited by any of the above described exemplary embodiments, but should be defined only in accordance with the following claims and their equivalents.

All documents cited herein, including journal articles or abstracts, published or corresponding U.S. or foreign patent applications, issued or foreign patents, or any other documents, are each entirely incorporated by reference herein, including all data, tables, figures, and text presented in the cited documents.

What is claimed is:

1. A method for treating arthritis in a subject, comprising administering about 430 mg to about 12.4 g of docosahexaenoic acid (DHA) per day in a dosage form to the subject in need thereof, wherein the dosage form is substantially free of eicosapentaenoic acid (EPA) and substantially free of non-alpha tocopherol.

2. The method of claim 1, wherein the EPA is less than about 3% (w/w) of the total fatty acid content of the dosage form.

3. The method of any of claims 1-2, wherein the EPA is less than about 0.1% (w/w) of the total fatty acid content of the dosage form.

4. The method of any of claims 1-3, wherein the EPA is not detectable in the dosage form.

5. The method of any of claims 1-4, wherein the dosage form is substantially free of docosapentaenoic acid 22:5n-6 (DPAn6).

6. The method of any of claims 1-5, wherein the DHA is derived from an algal source.

7. The method of claim 6, wherein the algal source is Crypthecodiunium cohnii, Thraustochytrium, or Schizochytrium sp.

8. The method of any of claims 1-7, wherein about 0.84 g to about 4 g of DHA is administered per day to the subject.

9. The method of claim 8, wherein about 0.84 g to about 1.5 g of DHA is administered per day to the subject.

10. The method of claim 8, wherein about 0.84 mg to about 1 g of DHA is administered per day to the subject.

11. The method of any of claims 1-10, wherein the DHA is administered once per day, twice per day, three times per day, four times per day, five times per day, six times per day, seven times per day, eight times per day, nine times per day, ten times per day, eleven times per day, or twelve times per day.

12. The method of any of claims 1-11, wherein the DHA is administered in a single dosage form.

13. The method of any of claims 1-12, wherein the dosage form comprises about 0.4 g to about 1 g of DHA.

14. The method of any of claims 1-13, wherein the dosage form has a total weight of about 0.2 g, about 0.35 g, about 0.4 g, about 0.45 g, about 0.5 g, about 0.55 g, about 0.6 g, about 0.65 g, about 0.7 g, about 0.75 g, about 0.8 g, about 0.85 g, about 0.9 g, about 0.95 g, about 1 g, or about 1.05 g.

15. The method of any of claims 1-14, wherein the DHA in the dosage form is about 30% to about 99.5% (w/w) of the total fatty acid content of the dosage form.

16. The method of any of claims 1-15, wherein the DHA in the dosage form is about 35% to about 65% (w/w) of the total fatty acid content of the dosage form.

17. The method of any of claims 1-16, wherein the dosage form is characterized by one or more of the following amounts of fatty acids or esters thereof:
(a) capric acid is about 1% or less;
(b) lauric acid is about 1% or less;
(c) myristic acid is about 1% or less;
(d) palmitic acid is about 1% or less;
(e) palmitoleic acid is about 1% or less;
(f) stearic acid is about 1% or less;
(g) oleic acid is about 1% or less;
(h) linoleic acid is about 1% or less;
(i) α-linolenic acid is about 1% or less;
(j) docosapentaenoic acid 22:5n-3 (DPAn3) is about 1% or less;
(k) docosapentaenoic acid 22:5n-6 (DPAn6) is about 1% or less; and
(l) 4,7,10,13,16,19,22,25 octacosaoctaenoic acid (C28:8) is about 1% or less of the total fatty acid content of the dosage form.

18. The method of any of claims 1-17, wherein the DHA in the dosage form comprises about 40% to about 50% (w/w) of the total weight of the dosage form.

19. The method of any of claims 1-18, wherein the DHA in the dosage form comprises about 40% to about 45% (w/w) of the total fatty acid content of the dosage form.

20. The method of any of claims 1-19, wherein the dosage form is characterized by one or more of the following amounts of fatty acid or esters thereof:
(a) capric acid (C10:0) is about 2% or less;
(b) lauric acid (C12:0) is about 6% or less;
(c) myristic acid (C14:0) is about 20% or less;
(d) palmitic acid (C16:0) is about 20% or less;
(e) palmitoleic acid (C16:1n-7) is about 3% or less;
(f) stearic acid (C18:0) is about 2% or less;
(g) oleic acid (C18:1n-9) is about 40% or less;
(h) linoleic acid (C18:2) is about 5% or less;
(i) arachidonic acid (C20:4) is about 0.1% or less;
(j) eicospentaenoic acid (C20:5) is about 3% or less;
(k) decosapentaenoic acid (22:5n-6) is about 0.1% or less;
(l) nervonic acid (C24:1) is about 2% or less; and
(m) others is about 3% or less;
of the total fatty acid content of the dosage form.

21. The method of any of claims 1-20, wherein the DHA in the dosage form comprises about 35% to about 45% (w/w) of the total weight of the dosage form.

22. The method any of claims 1-21, wherein the DHA in the dosage form comprises about 35% to about 45% (w/w) of the total fatty acid content of the dosage form.

23. The method of any of claims 1-22, wherein the dosage form is characterized by one or more of the following amounts of fatty acid or esters thereof:
(a) capric acid (C10:0) is about 0.01% or less;
(b) lauric acid (C12:0) is about 0.5% or less;
(c) myristic acid (C14:0) is about 12% or less;
(d) palmitic acid (C16:0) is about 28% or less;
(e) palmitoleic acid (C16:1n-7) is about 0.5% or less;
(f) stearic acid (C18:0) is about 2% or less;
(g) oleic acid (C18:1n-9) is about 8% or less;
(h) linoleic acid (C18:2) is about 2% or less;
(i) arachidonic acid (C20:4) is about 2% or less;
(j) eicospentaenoic acid (C20:5) is about 3% or less;
(k) decosapentaenoic acid (22:5n-6) is about 18% or less;
(l) nervonic acid (C24:1) is about 0.01% or less; and
(m) others is about 10% or less;
of the total fatty acid content of the dosage form.

24. The method of any of claims 1-23, wherein the DHA in the dosage form comprises about 55% to about 67% (w/w) of the total weight of the dosage form.

25. The method of any of claims 1-24, wherein the DHA in the dosage form comprises about 55% to about 67% (w/w) of the total fatty acid content of the dosage form.

26. The method of any of claims 1-25, wherein the dosage form is characterized by one or more of the following amounts of fatty acid or esters thereof:
(a) capric acid (C10:0) is about 2% or less;
(b) lauric acid (C12:0) is about 6% or less;
(c) myristic acid (C14:0) is about 20% or less;
(d) palmitic acid (C16:0) is about 15% or less;
(e) palmitoleic acid (C16:1n-7) is about 5% or less;
(f) stearic acid (C18:0) is about 2% or less;
(g) oleic acid (C18:1n-9) is about 20% or less;
(h) linoleic acid (C18:2) is about 2% or less;
(i) arachidonic acid (C20:4) is about 0.1% or less;
(j) eicospentaenoic acid (C20:5) is about 0.1% or less;
(k) decosapentaenoic acid (22:5n-6) is about 0.1% or less; and
(l) others is about 3% or less;
of the total fatty acid content of the dosage form.

27. The method of any of claims 1-26, wherein the DHA in the dosage form comprises about 67% to about 72% (w/w) of the total fatty acid content of the dosage form.

28. The method of any of claims 1-27, wherein the DHA in the dosage form comprises about 85% to about 96% (w/w) of the total weight of the dosage form.

29. The method of any of claims 1-28, wherein the DHA in the dosage form comprises about 85% to about 99.5% (w/w) of the total fatty acid content of the dosage form.

30. The method of any of claims 1-29, wherein the dosage form is characterized by one or more of the following amounts of fatty acid or esters thereof:
(a) capric acid (C10:0) is about 0.1% or less;
(b) lauric acid (C12:0) is about 0.1% or less;
(c) myristic acid (C14:0) is about 0.1% or less;
(d) palmitic acid (C16:0) is about 0.5% or less;
(e) palmitoleic acid (C16:1n-7) is about 0.5% or less;
(f) stearic acid (C18:0) is about 0.5% or less;
(g) oleic acid (C18:1n-9) is about 4% or less;
(h) linoleic acid (C18:2) is about 0.1% or less;
(i) arachidonic acid (C20:4) is about 0.1% or less;
(j) eicospentaenoic acid (C20:5) is about 0.1% or less;
(k) decosapentaenoic acid (22:5n-6) is about 3% or less; and
(l) others is about 1% or less;
of the total fatty acid content of the dosage form.

31. The method of any of claims 1-30, wherein the DHA is in the form of an ester.

32. The method of any of claims 1-31, wherein the DHA is in the form of a triglyceride.

33. The method of any of claims 1-32, wherein the ester is an alkyl ester.

34. The method of any of claims 1-33, wherein the alkyl ester is a methyl ester, ethyl ester, propyl ester, or combinations thereof.

35. The method of any of claims 1-34, wherein the dosage form is a unit dose comprising about 430 mg to about 480 mg of DHA ethyl ester.

36. The method of any of claims 1-35, wherein the dosage form is a unit dose comprising about 860 mg to about 950 mg of the DHA ethyl ester.

37. The method of any of claims 1-36, wherein the dosage form is a unit dose comprising comprises about 870 mg to about 930 mg of the DHA ethyl ester.

38. The method of any of claims 1-37, wherein the dosage form is administered once per day.

39. The method of any of claims 1-38, wherein the dosage form is administered once per day for the remainder of the subject's lifetime.

40. The method of any of claims 1-39, wherein the dosage form is administered once per day for about 1 year to about 10 years.

41. The method of any of claims 1-40, wherein the dosage form is administered once per day for about 1 month to about 12 months.

42. The method of any of claims 1-41, wherein the dosage form is administered once per day for at least 6 months.

43. The method of any of claims 1-42, wherein the dosage farm is an oral dosage foil i.

44. The method of claim 43, wherein the oral dosage form is a gelatin capsule or a caplet.

45. The method of claim 44, wherein the oral dosage form is a gelatin capsule.

46. The method of claim 45, wherein the gelatin capsule is a soft gelatin capsule.

47. The method of any of claims 45-46, wherein the gelatin capsule comprises a plasticizer, gelatin, and water.

48. The method of claim 47, wherein the plasticizer is glycerol or glycerin.

49. The method of any of claims 1-48, wherein the arthritis is selected from the group consisting of osteoarthritis, gouty arthritis, ankylosing spondylitis, psoriatic arthritis, reactive arthritis, rheumatoid arthritis, juvenile onset rheumatoid arthritis, infectious arthritis, inflammatory arthritis, septic arthritis, degenerative arthritis, arthritis mutilans, and lyme arthritis.

50. The method of any of claims 1-49, wherein the dosage form is administered in combination with an agent selected from the group consisting of nonsteroidal anti-inflammatory drugs (NSAIDs), cox-2 inhibitors, steroids, narcotic pain relievers, disease-modifying antirheumatic drugs (DMARDs), TNF blockers, biological response modifiers, chemotherapy medications, and gout medicines.

51. An oral dosage form consisting essentially of about 430 mg to about 480 mg of docosahexaenoic acid (DHA), wherein the dosage form is substantially free of eicosapentaenoic acid (EPA) and substantially free of non-alpha tocopherol.

52. An oral dosage form consisting essentially of about 860 mg to about 950 mg of docosahexaenoic acid (DHA), wherein the dosage form is substantially free of eicosapentaenoic acid (EPA) and substantially free of non-alpha tocopherol.

53. The dosage form of any of claims 51-52, wherein the dosage form is a gelatin capsule.

54. The dosage form of claim 53, wherein the gelatin capsule is a soft gelatin capsule.

55. The dosage form of any of claims 53-54, wherein the gelatin capsule comprises a plasticizer, gelatin, and water.

56. The dosage from of claim 55, wherein the plasticizer is glycerol or glycerin.

57. The dosage form of any of claims 51-56, wherein the dosage form comprises about 860 mg to about 950 mg DHA.

58. The dosage form of any of claims 51-57, wherein the dosage form comprises about 870 mg to about 930 mg DHA.

59. The dosage form of any of claims 51-58, wherein the DHA is derived from an algal source.

60. A kit comprising the dosage form of any of claims 51-59 and a label containing instructions to administer the dosage form once per day, twice per day, three times per day, four times per day, five times per day, six times per day, seven times per day, eight times per day, nine times per day, ten times per day, eleven times per day, or twelve times per day, to a subject in need thereof.
